SEQUENCE LISTING (10 sequences)

SEQ ID NO: 1 - DNA, 201 bp, Homo sapiens
  source 1..201: mol_type = genomic DNA
atgaaaagggattgtttgctttttatagatgaagaaactgaagctgagagatcaatatatccaggccacagagttcatgggttatgtaactgcaattagcdcacaagtcttgcatctctagaacctgtcttcaatgccatggcacgacactgccttaccattgaacctcttgtgtactgggacatgtgactttgattaaag

SEQ ID NO: 2 - DNA, 201 bp, Homo sapiens
  source 1..201: mol_type = genomic DNA
ctgaaattgattgagcttgatttttcatacaggagttatgtgaaaaaattatttgagcctttgtttatggaagagtcaaaagtattgtttgaagttaatantgaaattctaaactatgaatttttatattctattaaagtcacctcttccacttttccccatttcaagtgttctccagaattttaatgagtattagctggt

SEQ ID NO: 3 - DNA, 201 bp, Homo sapiens
  source 1..201: mol_type = genomic DNA
ttttaattctttattagtcatctttttggcatgtgggtccctgtcccttatctgtctcactcctgtgatctgtaggcatccactcaaaatgttcctcctgncccccatacactgccacagtctttcactggactatgacatgctcttccctcagatctcagctttctccaggtactgattgtccattactttactcacctc

SEQ ID NO: 4 - DNA, 201 bp, Homo sapiens
  source 1..201: mol_type = genomic DNA
aatgtcatatttattatttttccctgagccatcgggtctgcgcttggcacataatggaaactcatacaatgatttctaaggtactgacagtgaaggtgacngcctagatcctgaaggatgcatctgtgctttattagagaaaaagagaggaaaaatgataagcgaatcccatgaaagggaagaaaaatgagataaaacata

SEQ ID NO: 5 - DNA, 201 bp, Homo sapiens
  source 1..201: mol_type = genomic DNA
aagggaaactccttataaaatcatcagatctcatgagacttattcactaccatgagaacagtatgggggaaactgcccccatgattcaattatttcccacngggtccctcccacaacacatgagaattatgagagctacaatgcaagatgagatttgggtagagacacagccaaaccatatcatcccatcccagcccctcc

SEQ ID NO: 6 - DNA, 201 bp, Homo sapiens
  source 1..201: mol_type = genomic DNA
ccaatagtttactagtgcattaaaatcacaatacattacaaggtaattaactagagctgccactataaatagaaaaatagtcacagccttcactataaacnacttaataaaaataaggttctgaatcagcattcagcattatagtaattcctcttgccatacgtctcattaatcaggacttttttaaaaagcttcttctca

SEQ ID NO: 7 - DNA, 201 bp, Homo sapiens
  source 1..201: mol_type = genomic DNA
tgaactagagaaatgccaggcattaagtttcatgtgcaaattgaattatattcagcaaatccactcactgaaaatattttttaaagttcaaaataagcagntggttctgcctagtgcatctatattagaaataggctgtagccaaagatttactattacaaattggaaacattaacacctgctattatctagttaagtata

SEQ ID NO: 8 - DNA, 201 bp, Homo sapiens
  source 1..201: mol_type = genomic DNA
ttcaaagggtgttaaataaaggcttggtactgtcatgctctgaacattcttcttcaggtttgatgtgactggttgcagatccagtgataatgatgctcacrttgaatacagtggctccccgatgttgattctccccttgatcactgttccagagaagagaaatcactgttccagagaaaagaaagtcaccgggattagtat

SEQ ID NO: 9 - DNA, 201 bp, Homo sapiens
  source 1..201: mol_type = genomic DNA
aaatctgcaatattatttttctttataaaatcgacccagctgaaatattttctgatgagtttaaatgcatatgaactagtgctctagagactgtcttaatdgagtacttcattttgtactttattgaaatgtgtactttgaaaacattggtgaaatttgccatttcccacctggcatcagcaacagctggagcctggccca

SEQ ID NO: 10 - DNA, 201 bp, Homo sapiens
  source 1..201: mol_type = genomic DNA
tattatttttctttataaaatcgacccagctgaaatattttctgatgagtttaaatgcatatgaactagtgctctagagactgtcttaatggagtacttcrttttgtactttattgaaatgtgtactttgaaaacattggtgaaatttgccatttcccacctggcatcagcaacagctggagcctggcccatttacgatgg

(19) United States
(12) Patent Application Publication
(10) Pub. No.: US 2026/0071276 A1
(43) Pub. Date: Mar. 12, 2026

(54) GPCR LATROPHILIN-3 AS BIOMARKER FOR DETECTING INCREASED RISK FOR MILD BRAIN INJURY AND METHODS OF USE THEREOF FOR DIAGNOSIS AND TREATMENT OF THE SAME

(71) Applicant: THE CHILDREN'S HOSPITAL OF PHILADELPHIA, Philadelphia, PA (US)

(72) Inventors: Hakon HAKONARSON, Malvern, PA (US); Christina MASTER, Haverford, PA (US); Kristy ARBOGAST, Media, PA (US)

(73) Assignee: THE CHILDREN'S HOSPITAL OF PHILADELPHIA, Philadelphia, PA (US)

(21) Appl. No.: 19/109,790

(22) PCT Filed: Sep. 8, 2023
(86) PCT No.: PCT/US2023/073763

§ 371 Date: Mar. 7, 2025

Related U.S. Application Data
(60) Provisional application No. 63/375,001, filed on Sep. 8, 2022.

(51) Int. Cl. C12Q 1/6883

(52) U.S. Cl. CPC C12Q 1/6883; C12Q 2600/118; C12Q 2600/156

(57) ABSTRACT

Compositions and methods for diagnosis and treatment of increased risk for mild traumatic brain injury are disclosed.

### CROSS REFERENCE TO RELATED APPLICATION

This application claims priority to U.S. Provisional Application No. 63/375,001 filed Sep. 8, 2022, the entire contents being incorporated herein by reference as though set forth in full.

### INCORPORATION BY REFERENCE OF MATERIAL SUBMITTED IN ELECTRONIC FORM

The contents of the electronic sequence listing (SEQLIST.xml; Size: 11,692 bytes; and Date of Creation: Sep. 8, 2023) is herein incorporated by reference in its entirety.

### FIELD OF THE INVENTION

The present disclosure relates to a method for detecting an increased risk for mild traumatic brain injury or concussion in a subject in need thereof, by detecting one or both of a pair of single nucleotides polymorphisms present in the latrophilin-3 (ADGRL3) gene which, when present, confer a greater risk for concussive injury, particularly in subjects having African American ancestry. The present disclosure also provides novel agents that interfere with latrophilin 3 signaling for the treatment of mild traumatic brain injury.

### BACKGROUND OF THE INVENTION

Several publications and patent documents are cited throughout the specification in order to describe the state of the art to which this invention pertains. Each of these citations is incorporated herein by reference as though set forth in full.

The ADGRL3 gene (also known as the LPHN3) encodes a member of the latrophilin subfamily of G protein-coupled receptors (GPCR). Latrophilins may function in both cell adhesion and signal transduction. In experiments with non-human species, endogenous proteolytic cleavage within a cysteine-rich GPS (G-protein-coupled-receptor proteolysis site) domain resulted in two subunits (a large extracellular N-terminal cell adhesion subunit and a subunit with substantial similarity to the secretin/calcitonin family of GPCRs) being non-covalently bound at the cell membrane. Latrophilin-3 is also a regulator of synaptic function and maintenance in brain regions that mediate locomotor activity, attention, and memory for location and path. Variants of LPHN3 are associated with increased risk for attention deficit hyperactivity disorder (ADHD) in some patients. Some studies suggest that LPHN3 is involved in cognition as well as activity and attention. The evidence shows that LPHN3 plays a more significant role in neuroplasticity than previously appreciated.

A concussion is a “traumatically induced transient disturbance of brain function” associated with mild or moderate brain injury. Traumatic brain injuries have varying severity, ranging from mild, transient symptoms to extended periods of altered consciousness. Given the usually self-limited nature of symptoms associated with a concussion, the term mild traumatic brain injury (mTBI) is often used interchangeably to refer to a concussion, though concussions are technically a subset of mTBIs. Prognosis is usually good, and most patients experience complete resolution of symptoms. A concussion occurs as a result of either a direct or indirect injury to the head. Providers often consider a direct, traumatic blow to the head as a significant cause of a concussion. However, indirect traumatic forces elsewhere in the body can lead to an acute acceleration/deceleration injury to the brain, which can also lead to a concussion.

Diagnosis of a concussion remains an exclusively clinical diagnosis based on history and exam findings. However, there is no single pathognomonic finding or a minimum number of symptoms for diagnosing a concussion. Several standardized diagnostic tools can be employed in the pre-hospital setting following an acute head injury to assist in determining the presence of a concussion. It remains uncertain whether genetic susceptibility plays a role in the vulnerability or symptom severity of concussion. The present invention addresses these and other needs.

### SUMMARY OF THE INVENTION

In accordance with the invention, a method for identifying a subject at increased risk for concussion or mild traumatic brain injury (mTBI) is provided. An exemplary method entails detecting in a biological sample obtained from a subject a nucleic acid encoding the risk allele in present in the ADGRL3 gene listed in Table 2 associated with an increased risk of mTBI; and diagnosing said subject as being at an increased risk for mTBI or concussion. In preferred embodiments, the nucleic acid encodes the risk allele present in rs1470721. The sample can be a biological sample derived from said subject prior to the brain injury or within about 0.1 to about 8 hours of a head injury. In certain approaches, the step of detecting in the nucleic acid a single nucleotide polymorphism (SNP) in said ADGRL3 gene comprises performance of a process selected from the group consisting of detection of specific hybridization, measurement of allele size, restriction fragment length polymorphism analysis, allele-specific hybridization analysis, single base primer extension reaction, and sequencing of an amplified polynucleotide. In some embodiments, the process is performed on a solid support. Suitable solid supports for this purpose include without limitation, a strip, a glass, a silicon, a polymer, a bead, or a nanoparticle.

The invention also provides a method of treating concussion or mild traumatic brain injury (mTBI) with an agent that modulates ADGRL3 activity. An exemplary method comprises detecting in a biological sample obtained from a subject suspected of having or being at increased risk for concussion or mTBI, a nucleic acid encoding one or more SNPs comprising risk alleles present in the ADGRL3 gene listed in Table 2; diagnosing said subject as being at an increased risk for mTBI or concussion and administering to said subject a therapeutically effective amount of a compound that modulates the activity or level of ADGRL3, thereby treating said subject. In preferred embodiments, the agents include one or more of the glutamatergic or ionotropic agents listed in Table 3. In certain embodiments, the nucleic acid encodes the risk allele present in rs1470721. In a preferred embodiment, the gluatamatergic modulator is fasoracetam or a pharmaceutically acceptable salt, ester, prodrug, metabolite, polymorph or solvate thereof.

Also disclosed is a method for protecting a subject from mTBI wherein said subject participates in a sports activity associated with a high incidence of mTBI, comprising detecting in a biological sample obtained from a subject suspected of having or being at increased risk for concussion or mTBI, a nucleic acid encoding one or more SNPs comprising risk alleles present in the ADGRL3 gene listed in Table 2; and administering an effective amount of a glutamatergic or ionotropic modulator to said subject, prior to participation in said activity, thereby reducing the risk of mTBI in said subject.

### BRIEF DESCRIPTION OF THE DRAWINGS

FIG. 1: Manhattan plot of the results of the CHOP trans-ancestral meta-analysis. P-values are shown as the −log 10 of the p-value.

FIG. 2: Quantile-Quantile of the results of the CHOP trans-ancestral meta-analysis.

FIG. 3: Manhattan plot of the results of the CHOP European-ancestry only meta-analysis. P-values are shown as the −log 10 of the p-value.

FIG. 4: Quantile-Quantile of the results of the CHOP European-ancestry only meta-analysis.

FIG. 5: Manhattan plot of the results of the CHOP African-ancestry only meta-analysis. P-values are shown as the −log 10 of the p-value.

FIG. 6: Quantile-Quantile of the results of the CHOP African-ancestry only meta-analysis.

FIG. 7: Manhattan plot of the results of the Multi-center trans-ancestral meta-analysis. P-values are shown as the −log 10 of the p-value.

FIG. 8: Quantile-Quantile of the results of the Multi-center trans-ancestral meta-analysis.

FIG. 9: The linkage disequilibrium and the recombination rate are shown in the 1000 genomes project ASW population in the region of rs1470721 (using rs1470721 as the index SNP).

Variants are labeled with numbers corresponding to their RegulomeDB score.

### DETAILED DESCRIPTION OF THE INVENTION

Concussion is a relatively common traumatic brain injury in children and adolescents that affects brain functioning, presenting with variety of symptoms, including but not limited to transient loss of consciousness, memory loss, headaches, difficulty with concentration and mood changes of varying duration. While the causes for the injury, such as motor vehicle collisions, sports injuries, falls or bicycle accidents are usually apparent, we wished to determine if genetic susceptibility plays a role in the vulnerability or symptom severity of concussion. To address this issue, we performed a genome wide association study using the lifetime occurrence (yes/no) of a concussion as the primary trait. Eight groups of patients were analyzed. Six groups were children and adolescents from The Children's Hospital of Philadelphia (CHOP), 1 group was from the UK Biobank (UKB) and 1 group was from the Finngen Biobank (FB). Collectively, the CHOP cohorts included 2,314 cases (1,294 Europeans vs. 1,020 African Americans) and 113,395 controls (75,652 Europeans vs. 37,743 African Americans). The FB dataset included 8,136 cases and 113,935 controls and defined concussion using ICD10 code S060, ICD9 code 850 or ICD8 code 850. The UKB dataset included 245 cases and 405,554 controls, and defined concussion using Phecode ID number 817. This included ICD10 code S06 and ICD9 code 850. SAIGE was used for association in each cohort to control for case-control imbalances and a meta-regression (MR-MEGA) meta-analysis was used for a trans-ancestral analysis.

A single genome wide significant locus was uncovered on chromosome 4, (rs1470721, p=2.26×10⁻⁸). Two SNPs at the locus attained genome wide significance and multiple other SNPs at the locus were just below genome wide significance further supporting the association. There was a significant association at this locus with ancestry (p=3.53×10⁻⁹) as the association was most robustly driven by the African American cohorts from CHOP. The index SNP at the locus is intronic to the ADGRL3 gene encoding latrophilin-3 also known as the LPHN3 gene. The index SNP also shows nominal significance (p=0.046) as an eQTL to ADGRL3 in brain anterior cingulate cortex (Brodmann area 24) (GTEx Analysis Release V8 (dbGaP Accession phs000424.v8.p2)). In contrast, previous association at the ApoD locus continues to be in the suggestive range of association significance.

ADGRL3 is a synaptic adhesion molecule that is one of the principal components of the molecular machinery that connects pre- and postsynaptic neurons, facilitates glutamatergic transmission, controls synaptic plasticity and empowers intersecting neural circuits to process and refine information involving the glutamate neurotransmitter system also involving regulation of excitatory synapse development. Studies using affinity chromatography and mass spectrometry demonstrate that latrophilin-3 (LPHN3) ectodomains interact with high affinity in trans and that interference with this interaction using soluble recombinant LPHN3, LPHN3 shRNA, or FLRT3 shRNA reduces excitatory synapse density in cultured neurons. In addition, previous studies show that reducing FLRT3 levels with shRNA in vivo decreases afferent input strength and dendritic spine number in dentate granule cells, suggesting that LPHN3 and its ligand FLRT3 may play an important role in glutamatergic synapse development. Thus, ADGRL3 is a G protein-coupled receptor associated with attention deficit hyperactivity disorder and human cognitive function that we for the first time are reporting as a genome-wide significant locus in children and adolescents with concussion, an association most significantly driven by concussion patients of African American ancestry. Taken together, these findings suggest that the glutamatergic modulator, fasoracetam and related racetam compounds, should provide an effective therapy for patients who have sustained concussion injury.

### Definitions

A “single nucleotide polymorphism (SNP)” refers to a change in which a single base in the DNA differs from the usual base at that position. These single base changes are called SNPs or “snips.” Millions of SNP's have been cataloged in the human genome. Some SNPs such as that which causes sickle cell are responsible for disease. Other SNPs are normal variations in the genome. Certain SNPs can indicate an increased risk of a given condition.

The term “genetic alteration” as used herein refers to a change from the wild-type, normal or protective allele, or reference sequence of one or more nucleic acid molecules. Genetic alterations can also include without limitation, base pair substitutions, additions and deletions of at least one nucleotide from a nucleic acid molecule of known sequence.

“Target nucleic acid” as used herein refers to a previously defined region of a nucleic acid present in a complex nucleic acid mixture wherein the defined wild-type region contains at least one known nucleotide variation, which may or may not be associated with increased risk of mild traumatic brain injury or concussion. The nucleic acid molecule may be isolated from a natural source by cDNA cloning or subtractive hybridization or synthesized manually. The nucleic acid molecule may be synthesized manually by the triester synthetic method or by using an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated.

The term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any mild traumatic brain injury (MTBI) specific marker gene or nucleic acid, but does not hybridize to other nucleotides. Also polynucleotides which “specifically hybridizes” may hybridize only to a specific MTBI or concussion marker. Appropriate conditions enabling specific hybridization of single stranded nucleic acid molecules of varying complementarity are well known in the art. For instance, one common formula for calculating the stringency conditions required to achieve hybridization between nucleic acid molecules of a specified sequence homology is set forth in (Sambrook et al., Molecular Cloning, Cold Spring Harbor Laboratory (1989).

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product. Probes and primers having the appropriate sequence homology which specifically hybridized to SNP containing nucleic acids are useful in the detecting the presence of such nucleic acids in biological samples.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Those skilled in the art will recognize that a nucleic acid vector can contain nucleic acid elements other than the promoter element and the MTBI specific marker gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

“Sample” or “patient sample” or “biological sample” generally refers to a sample, which may be tested for a particular molecule, preferably an autism specific marker molecule, such as a marker shown in the tables and figures provided herein. Samples may include but are not limited to cells, body fluids, including blood, serum, plasma, urine, saliva, tears, pleural fluid and the like.

A “biomarker” used herein refers to a molecular indicator of a specific biological property; a biochemical feature or facet that can be used to detect mild traumatic brain injury. “Biomarker” encompasses, without limitation, proteins, nucleic acids, and metabolites, together with their polymorphisms (e.g., single nucleotide polymorphisms (SNP), mutants, isoform variants, related metabolites, derivatives, precursors including nucleic acids and pro-proteins, cleavage products, protein-ligand complexes, post-translationally modified variants (such as cross-linking or glycosylation), fragments, and degradation products, as well as any multi-unit nucleic acid, protein, and glycoprotein structures comprised of any of the biomarkers as constituent subunits of the fully assembled structure, and other analytes or sample-derived measures.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The term “modulate” as used herein refers to increasing/promoting or decreasing/inhibiting a particular cellular, biological or signaling function associated with the normal activities of the SNP containing molecules described herein or the proteins encoded thereby. For example, the term modulate refers to the ability of a test compound or test agent to interfere with signaling or activity of a gene or protein of the present invention in vivo and in vitro.

As used herein, “treatment” is an approach for obtaining beneficial or desired results, including clinical results. For purposes of this disclosure, beneficial or desired clinical results include, but are not limited to, alleviation or amelioration of symptoms (e.g., amnesia, nausea, vomiting, headache, diplopia, dizziness, sleepiness, confusion, and/or disorientation/sensation of “fogginess”), diminishment of extent of head injury, stabilized (i.e., not worsening) state of head injury, delay or slowing of head injury progression, amelioration or palliation of the head injury state, and recovery (whether partial or total), whether detectable or undetectable. The patient can be treated before, during or after the head injury.

The term “subject” as used herein includes all members of the animal kingdom prone to suffering from the indicated disorder. In some aspects, the subject is a mammal, and in some aspects, the subject is a human of general population. The methods are also applicable to companion animals such as dogs and cats as well as livestock such as cows, horses, sheep, goats, pigs, and other domesticated and wild animals. In some cases, the subject is suffering from one or more following symptoms: amnesia, nausea, vomiting, headache, diplopia, dizziness, sleepiness, confusion, disorientation, and sensation of “fogginess.

### Methods of Using mTBI-Associated SNPS for Diagnosing an Increased Susceptibility for mTBI

The present invention provides methods of diagnosing mTBI in a patient or methods for identifying a patient having an increased risk for mTBI. Diagnosis, as used herein, includes not only the initial identification of mTBI associated with the genetic alterations described herein in a patient but confirmatory testing, or screening in patients who have previously been identified as having or likely to have mTBI. The methods include the steps of providing a biological sample from the patient, detecting the presence of SNPs or any other mTBI associated markers present in the biological sample, preferably a tissue and/or blood plasma sample, and determining if the patient has a greater likelihood of mTBI based on the amount and/or type of mTBI marker expression level determined relative to those expression levels identified in patient cohorts of known outcome. A patient has a greater likelihood of having mTBI when the sample has a SNP marker expression profile associated with patients previously diagnosed with mTBI. The compositions and methods of the invention are useful for the prognosis and diagnosis and management of mTBI

In another aspect, the patient sample may have been previously genotyped and thus the genetic expression profile in the sample may be available to the clinician. Accordingly, the method may entail storing reference mTBI associated marker sequence information in a database, i.e., those SNPs statistically associated with a more favorable or less favorable prognosis, and performance of comparative genetic analysis on the computer, thereby identifying those patients having increased risk mTBI.

mTBI-related SNP-containing nucleic acids, including but not limited to those listed below may be used for a variety of purposes in accordance with the present invention. mTBI-SNP-containing DNA, RNA, or fragments thereof may be used as probes to detect the presence of and/or expression of mTBI specific markers. Methods in which mTBI specific marker nucleic acids may be utilized as probes for such assays include, but are not limited to: (1) in situ hybridization; (2) Southern hybridization (3) northern hybridization; and (4) assorted amplification reactions such as polymerase chain reactions (PCR).

Further, assays for detecting mTBI-associated SNPs may be conducted on any type of biological sample, including but not limited to body fluids (including blood, urine, serum, gastric lavage, cerebral spinal fluid), any type of cell (such as brain cells, white blood cells, mononuclear cells, fetal cells in maternal circulation) or body tissue.

Clearly, mTBI-associated SNP-containing nucleic acids, vectors expressing the same, mTBI SNP-containing marker proteins and anti-mTBI specific marker antibodies of the invention can be used to detect mTBI associated SNPs in body tissue, cells, or fluid, and alter mTBI SNP-containing marker protein expression for purposes of assessing the genetic and protein interactions involved in the development of mTBI.

In most embodiments for screening for mTBI-associated SNPs, the mTBI-associated SNP-containing nucleic acid in the sample will initially be amplified, e.g. using PCR, to increase the amount of the templates as compared to other sequences present in the sample. This allows the target sequences to be detected with a high degree of sensitivity if they are present in the sample. This initial step may be avoided by using highly sensitive array techniques that are important in the art.

Alternatively, new detection technologies can overcome this limitation and enable analysis of small samples containing as little as 1 g of total RNA. Using Resonance Light Scattering (RLS) technology, as opposed to traditional fluorescence techniques, multiple reads can detect low quantities of mRNAs using biotin labeled hybridized targets and anti-biotin antibodies. Another alternative to PCR amplification involves planar wave guide technology (PWG) to increase signal-to-noise ratios and reduce background interference. Both techniques are commercially available from Qiagen Inc. (USA).

Any of the aforementioned techniques may be used to detect or quantify mTBI-associated SNP marker expression and accordingly, diagnose mTBI.

### Kits and Articles of Manufacture

Any of the aforementioned products can be incorporated into a kit which may contain a mTBI-SNP specific marker polynucleotide or one or more such markers immobilized on a Gene Chip, an oligonucleotide, a polypeptide, a peptide, an antibody, a label, marker, reporter, a pharmaceutically acceptable carrier, a physiologically acceptable carrier, instructions for use, a container, a vessel for administration, an assay substrate, or any combination thereof.

The following materials and methods are provided to facilitate the practice of the invention.

### Cohorts

The meta-analysis combined genome-wide association studies utilizing samples from the Children's Hospital of Philadelphia (CHOP), the UK BioBank (UKBB), and the Finngen BioBank data release 4. The phenotype was lifetime occurrence of concussion. The UKBiobank analysis (245 cases, 405554 controls) defined concussion using Phecode ID number 817.[1] The UKBioBank summary statistics were downloaded from the world wide web at .leelabsg.org/resources. This included ICD10 code S06 and ICD9 code 850.9. Exclusions include icd9 codes 816, 818, 818.2, and 819. The Finngen Biobank r4 (8136 cases, 113935 controls) defined concussion using ICD10 code S060, ICD9 code 850 or icd8 code 850. The CHOP cohorts included 2314 cases (1294 European, 1020 African) and 113395 controls (75652 European, 37743 African). Inclusion criteria for CHOP patients is a concussion-related ICD-9-CM code listed in Tables 1A, 1B, or 1C. Patients are excluded as cases if they have any of the non-concussion TBI code listed in Table 1D for the same visit as an inclusion code is recorded. All subjects gave informed consent before inclusion in the study.

TABLE 1A
Concussion-related ICD-9-CM codes

| | |
|---|---|
| 850 | Concussion |
| 850 | Concussion with no loss of consciousness |
| 850.1 | Concussion with brief loss of consciousness |
| 850.1 | Concussion with brief loss of consciousness |
| 850.11 | Concussion, with loss of consciousness of 30 minutes or less |
| 850.5 | Concussion with loss of consciousness of unspecified duration |
| 850.9 | Concussion, unspecified |

TABLE 1B
Skull fracture with concussion-related ICD-9-CM codes

| | |
|---|---|
| 800.09 | Closed fracture of vault of skull without mention of intracranial injury, with concussion, unspecified |
| 800.59 | Open fracture of vault of skull without mention of intracranial injury, with concussion, unspecified |
| 801.09 | Closed fracture of base of skull without mention of intra cranial injury, with concussion, unspecified |
| 801.39 | Closed fracture of base of skull with concussion, unspecified |
| 801.59 | Open fracture of base of skull without mention of intracranial injury, with concussion, unspecified |
| 803.09 | Other and unqualified skull fractures with concussion, unspecified |
| 803.59 | Other open skull fracture without mention of intracranial injury, with concussion, unspecified |
| 804.09 | Closed fractures involving skull of face with other bones, without mention of intracranial injury, with concussion, unspecified |

TABLE 1C
Skull fracture with brief loss of consciousness (all LOC <1 hour)

| | |
|---|---|
| 800.02 | fracture of vault of skull w/brief (less than 1 hour) LOC |
| 800.52 | Open fracture of vault of skull without mention of intracranial injury, |
| | with brief [less than one hour] loss of consciousness |
| 801.02 | Closed fracture of base of skull without mention of intra cranial injury, |
| | with brief [less than one hour] loss of consciousness |
| 801.52 | Open fracture of base of skull without mention of intracranial injury |
| | with brief [less than one hour] loss of consciousness |
| 801.56 | Open fracture of base of skull without mention of intracranial injury |
| | with loss of consciousness of unspecified duration |
| 803.02 | Other and unqualified skull fractures with brief [less than one hour] loss of consciousness |
| 803.52 | Other open skull fracture without mention of intracranial injury with |
| | brief [less than one hour] loss of consciousness |
| 804.02 | Closed fractures involving skull or face with other bones, without mention |
| | of intracranial injury, with brief [less than one hour] loss of consciousness |
| 804.52 | Open fractures involving skull or face with other bones, without mention of |
| | intracranial injury, with brief [less than one hour] loss of consciousness |

TABLE 1D
Exclusion criteria for studies described in Example 1
Non-concussion TBI ICD-9-CM codes.

| | |
|---|---|
| 800.03 | fracture of vault of skull w/moderate (1-24 hrs) LOC |
| 800.05 | Closed fracture of vault of skull without mention of intracranial injury, with prolonged [more than |
| | 24 hours] loss of consciousness, without return to pre-existing conscious level |
| 800.1 | closed fracture of vault of skull w/cerebral laceration and contusion and |
| | unspecified state of consciousness |
| 800.1 | Closed fracture of vault of skull with cerebral laceration and |
| | contusion |
| 800.11 | closed fracture of vault of skull w/cerebral laceration and contusion |
| | and no LOC |
| 800.12 | closed fracture of vault of skull w/cerebral laceration and contusion and |
| | brief (<1 hr) LOC |
| 800.13 | Closed fracture of vault of skull with cerebral laceration and contusion, with |
| | moderate [1-24 hours] loss of consciousness |
| 800.14 | closed fracture of vault of skull w/cerebral laceration and contusion and with prolonged [more |
| | than 24 hours] loss of consciousness and return to pre-existing conscious |
| 800.15 | closed fracture of vault of skull w/cerebral laceration and contusion and with prolonged [more |
| | than 24 hours] loss of consciousness, without return to pre-existing conscious level |
| 800.16 | closed fracture of vault of skull w/cerebral laceration and contusion and with loss of |
| | consciousness of unspecified duration |
| 800.19 | closed fracture of vault of skull w/cerebral laceration and contusion with |
| | concussion, unspecified |
| 800.2 | Closed fracture of vault of skull with subarachnoid subdural and |
| | extradural hemorrhage |
| 800.2 | Closed fracture of vault of skull with subarachnoid, subdural, and extradural |
| | hemorrhage, unspecified state of consciousness |
| 800.21 | Closed fracture of vault of skull with subarachnoid, subdural, and extradural |
| | hemorrhage, with no loss of consciousness |
| 800.22 | Closed fracture of vault of skull with subarachnoid, subdural, and extradural hemorrhage, |
| | with brief [less than one hour] loss of consciousness |
| 800.23 | Closed fracture of vault of skull with subarachnoid, subdural, and extradural hemorrhage, |
| | with moderate [1-24 hours] loss of consciousness |
| 800.24 | Closed fracture of vault of skull with subarachnoid, subdural, and extradural hemorrhage, with |
| | prolonged [more than 24 hours] loss of consciousness and return to pre-existing conscious level |
| 800.25 | Closed fracture of vault of skull with subarachnoid, subdural, and extradural hemorrhage, with |
| | prolonged [more than 24 hours] loss of consciousness, without return to pre-existing conscious |
| | level |
| 800.26 | Closed fracture of vault of skull with subarachnoid, subdural, and extradural hemorrhage, |
| | with loss of consciousness of unspecified duration |
| 800.29 | Closed fracture of vault of skull with subarachnoid, subdural, and extradural |
| | hemorrhage, with concussion, unspecified |
| 800.3 | Closed fracture of vault of skull with other and unspecified intracranial |
| | hemorrhage |
| 800.3 | Closed fracture of vault of skull with other and unspecified intracranial |
| | hemorrhage, unspecified state of consciousness |
| 800.31 | Closed fracture of vault of skull with other and unspecified intracranial |
| | hemorrhage w/no loss of consciousness |
| 800.32 | Closed fracture of vault of skull with other and unspecified intracranial hemorrhage with |
| | brief [less than one hour] loss of consciousness |
| 800.33 | Closed fracture of vault of skull with other and unspecified intracranial hemorrhage |
| | with moderate [1-24 hours] loss of consciousness |
| 800.34 | Closed fracture of vault of skull with other and unspecified intracranial hemorrhage with |
| | prolonged [more than 24 hours] loss of consciousness and return to pre-existing conscious level |
| 800.35 | Closed fracture of vault of skull with other and unspecified intracranial hemorrhage with |
| | prolonged [more than 24 hours] loss of consciousness, without return to pre-existing conscious |
| | level |
| 800.36 | Closed fracture of vault of skull with other and unspecified intracranial hemorrhage |
| | with loss of consciousness of unspecified duration |
| 800.39 | Closed fracture of vault of skull with other and unspecified intracranial |
| | hemorrhage with with concussion, unspecified |
| 800.53 | Open fracture of vault of skull without mention of intracranial injury with |
| | moderate [1-24 hours] loss of consciousness |
| 800.55 | Open fracture of vault of skull without mention of intracranial injury, with prolonged [more than |
| | 24 hours] loss of consciousness, without return to pre-existing conscious level |
| 800.6 | Open fracture of vault of skull with cerebral laceration and |
| | contusion |
| 800.6 | Open fracture of vault of skull with cerebral laceration and contusion, |
| | unspecified state of consciousness |
| 800.61 | Open fracture of vault of skull with cerebral laceration and contusion with |
| | no loss of consciousness |
| 800.62 | Open fracture of vault of skull with cerebral laceration and contusion with brief |
| | [less than one hour] loss of consciousness |
| 800.63 | Open fracture of vault of skull with cerebral laceration and contusion with |
| | moderate [1-24 hours] loss of consciousness |
| 800.64 | Open fracture of vault of skull with cerebral laceration and contusion, with prolonged [more than |
| | 24 hours] loss of consciousness and return to pre-existing conscious level |
| 800.65 | Open fracture of vault of skull with cerebral laceration and contusion with prolonged [more than |
| | 24 hours] loss of consciousness, without return to pre-existing conscious level |
| 800.66 | Open fracture of vault of skull with cerebral laceration and contusion with loss of |
| | consciousness of unspecified duration |
| 800.69 | Open fracture of vault of skull with cerebral laceration and contusion, with |
| | concussion, unspecified |
| 800.7 | Open fracture of vault of skull with subarachnoid subdural and |
| | extradural hemorrhage |
| 800.7 | Open fracture of vault of skull with subarachnoid subdural and |
| | extradural hemorrhage |
| 800.71 | Open fracture of vault of skull with subarachnoid, subdural, and extradural |
| | hemorrhage, with no loss of consciousness |
| 800.72 | Open fracture of vault of skull with subarachnoid, subdural, and extradural hemorrhage, |
| | with brief [less than one hour] loss of consciousness |
| 800.73 | Open fracture of vault of skull with subarachnoid, subdural, and extradural hemorrhage, |
| | with moderate [1-24 hours] loss of consciousness |
| 800.74 | Open fracture of vault of skull with subarachnoid, subdural, and extradural hemorrhage, with |
| | prolonged [more than 24 hours] loss of consciousness and return to pre-existing conscious level |
| 800.75 | Open fracture of vault of skull with subarachnoid, subdural, and extradural hemorrhage, with |
| | prolonged [more than 24 hours] loss of consciousness, without return to pre-existing conscious |
| | level |
| 800.76 | Open fracture of vault of skull with subarachnoid, subdural, and extradural hemorrhage, |
| | with loss of consciousness of unspecified duration |
| 800.79 | Open fracture of vault of skull with subarachnoid, subdural, and extradural |
| | hemorrhage, with concussion, unspecified |
| 800.8 | Open fracture of vault of skull with other and unspecified intracranial |
| | hemorrhage |
| 800.8 | Open fracture of vault of skull with other and unspecified intracranial |
| | hemorrhage |
| 800.81 | Open fracture of vault of skull with other and unspecified intracranial |
| | hemorrhage with no loss of consciousness |
| 800.82 | Open fracture of vault of skull with other and unspecified intracranial hemorrhage, |
| | with brief [less than one hour] loss of consciousness |
| 800.84 | Open fracture of vault of skull with other and unspecified intracranial hemorrhage, with prolonged |
| | [more than 24 hours] loss of consciousness and return to pre-existing conscious level |
| 800.85 | Open fracture of vault of skull with other and unspecified intracranial hemorrhage with prolonged |
| | [more than 24 hours] loss of consciousness, without return to pre-existing conscious level |
| 800.86 | Open fracture of vault of skull with other and unspecified intracranial hemorrhage |
| | with loss of consciousness of unspecified duration |
| 800.89 | Open fracture of vault of skull with other and unspecified intracranial |
| | hemorrhage, with concussion, unspecified |
| 801.03 | Closed fracture of base of skull without mention of intra cranial injury, with |
| | moderate [1-24 hours] loss of consciousness |
| 801.04 | Closed fracture of base of skull without mention of intra cranial injury, with prolonged [more than |
| | 24 hours] loss of consciousness and return to pre-existing conscious level |
| 801.05 | Closed fracture of base of skull without mention of intra cranial injury, with prolonged [more than |
| | 24 hours] loss of consciousness, without return to pre-existing conscious level |
| 801.1 | Closed fracture of base of skull with cerebral laceration and |
| | contusion |
| 801.1 | Closed fracture of base of skull with cerebral laceration and contusion, |
| | unspecified state of consciousness |
| 801.11 | Closed fracture of base of skull with cerebral laceration and contusion with |
| | with no loss of consciousness |
| 801.12 | Closed fracture of base of skull with cerebral laceration and contusion with with brief |
| | [less than one hour] loss of consciousness |
| 801.13 | Closed fracture of base of skull with cerebral laceration and contusion with |
| | moderate [1-24 hours] loss of consciousness |
| 801.14 | Closed fracture of base of skull with cerebral laceration and contusion with prolonged [more than |
| | 24 hours] loss of consciousness and return to pre-existing conscious level |
| 801.15 | Closed fracture of base of skull with cerebral laceration and contusion with prolonged [more than |
| | 24 hours] loss of consciousness, without return to pre-existing conscious level |
| 801.16 | Closed fracture of base of skull with cerebral laceration and contusion with loss |
| | of consciousness of unspecified duration |
| 801.19 | Closed fracture of base of skull with cerebral laceration and contusion with |
| | concussion, unspecified |
| 801.2 | Closed fracture of base of skull with subarachnoid subdural and |
| | extradural hemorrhage |
| 801.2 | Closed fracture of base of skull with subarachnoid, subdural, and extradural |
| | hemorrhage, unspecified state of consciousness |
| 801.21 | Closed fracture of base of skull with subarachnoid, subdural, and extradural |
| | hemorrhage, with no loss of consciousness |
| 801.22 | Closed fracture of base of skull with subarachnoid, subdural, and extradural hemorrhage, |
| | with brief [less than one hour] loss of consciousness |
| 801.23 | Closed fracture of base of skull with subarachnoid, subdural, and extradural hemorrhage, |
| | with moderate [1-24 hours] loss of consciousness |
| 801.24 | Closed fracture of base of skull with subarachnoid, subdural, and extradural hemorrhage, with |
| | prolonged [more than 24 hours] loss of consciousness and return to pre-existing conscious level |
| 801.25 | Closed fracture of base of skull with subarachnoid, subdural, and extradural hemorrhage, with |
| | prolonged [more than 24 hours] loss of consciousness, without return to pre-existing conscious |
| | level |
| 801.26 | Closed fracture of base of skull with subarachnoid, subdural, and extradural hemorrhage, |
| | with loss of consciousness of unspecified duration |
| 801.29 | Closed fracture of base of skull with subarachnoid, subdural, and extradural |
| | hemorrhage, with concussion, unspecified |
| 801.3 | Closed fracture of base of skull with other and unspecified intracranial |
| | hemorrhage |
| 801.3 | Closed fracture of base of skull with other and unspecified intracranial |
| | hemorrhage, unspecified state of consciousness |
| 801.31 | Closed fracture of base of skull with other and unspecified intracranial |
| | hemorrhage with no loss of consciousness |
| 801.32 | Closed fracture of base of skull with other and unspecified intracranial hemorrhage with no loss of |
| | consciousness with brief [less than one hour] loss of consciousness |
| 801.33 | Closed fracture of base of skull with other and unspecified intracranial hemorrhage |
| | with moderate [1-24 hours] loss of consciousness |
| 801.34 | Closed fracture of base of skull with other and unspecified intracranial hemorrhage, with |
| | prolonged [more than 24 hours] loss of consciousness and return to pre-existing conscious level |
| 801.35 | Closed fracture of base of skull with other and unspecified intracranial hemorrhage, with |
| | prolonged [more than 24 hours] loss of consciousness, without return to pre-existing conscious |
| | level |
| 801.36 | Closed fracture of base of skull with other and unspecified intracranial hemorrhage |
| | with loss of consciousness of unspecified duration |
| 801.54 | Open fracture of base of skull without mention of intracranial injury, with prolonged [more than 24 |
| | hours] loss of consciousness and return to pre-existing conscious level |
| 801.55 | Open fracture of base of skull without mention of intracranial injury, with prolonged [more than 24 |
| | hours] loss of consciousness, without return to pre-existing conscious level |
| 801.6 | Open fracture of base of skull with cerebral laceration and |
| | contusion |
| 801.6 | Open fracture of base of skull with cerebral laceration and contusion, |
| | unspecified state of consciousness |
| 801.61 | Open fracture of base of skull with cerebral laceration and contusion with no |
| | loss of consciousness |
| 801.62 | Open fracture of base of skull with cerebral laceration and contusion, with brief [less |
| | than one hour] loss of consciousness |
| 801.63 | Open fracture of base of skull with cerebral laceration and contusion with |
| | moderate [1-24 hours] loss of consciousness |
| 801.64 | Open fracture of base of skull with cerebral laceration and contusion, with prolonged [more than |
| | 24 hours] loss of consciousness and return to pre-existing conscious level |
| 801.65 | Open fracture of base of skull with cerebral laceration and contusion with prolonged [more than 24 |
| | hours] loss of consciousness, without return to pre-existing conscious level |
| 801.66 | Open fracture of base of skull with cerebral laceration and contusion with loss of |
| | consciousness of unspecified duration |
| 801.69 | Open fracture of base of skull with cerebral laceration and contusion, with |
| | concussion, unspecified |
| 801.7 | Open fracture of base of skull with subarachnoid subdural and |
| | extradural hemorrhage |
| 801.71 | Open fracture of base of skull with subarachnoid, subdural, and extradural |
| | hemorrhage, with no loss of consciousness |
| 801.72 | Open fracture of base of skull with subarachnoid, subdural, and extradural hemorrhage, |
| | with brief [less than one hour] loss of consciousness |
| 801.73 | Open fracture of base of skull with subarachnoid, subdural, and extradural hemorrhage, |
| | with moderate [1-24 hours] loss of consciousness |
| 801.74 | Open fracture of base of skull with subarachnoid, subdural, and extradural hemorrhage, with |
| | prolonged [more than 24 hours] loss of consciousness and return to pre-existing conscious level |
| 801.75 | Open fracture of base of skull with subarachnoid, subdural, and extradural hemorrhage, with |
| | prolonged [more than 24 hours] loss of consciousness, without return to pre-existing conscious |
| | level |
| 801.76 | Open fracture of base of skull with subarachnoid, subdural, and extradural hemorrhage, |
| | with loss of consciousness of unspecified duration |
| 801.79 | Open fracture of base of skull with subarachnoid, subdural, and extradural |
| | hemorrhage, with concussion, unspecified |
| 801.8 | Open fracture of base of skull with other and unspecified intracranial |
| | hemorrhage |
| 801.81 | Open fracture of base of skull with other and unspecified intracranial |
| | hemorrhage, with no loss of consciousness |
| 801.82 | Open fracture of base of skull with other and unspecified intracranial hemorrhage, |
| | with brief [less than one hour] loss of consciousness |
| 801.83 | Open fracture of base of skull with other and unspecified intracranial hemorrhage |
| | with moderate [1-24 hours] loss of consciousness |
| 801.85 | Open fracture of base of skull with other and unspecified intracranial hemorrhage, with prolonged |
| | [more than 24 hours] loss of consciousness, without return to pre-existing conscious level |
| 801.86 | Open fracture of base of skull with other and unspecified intracranial hemorrhage |
| | with loss of consciousness of unspecified duration |
| 801.89 | Open fracture of base of skull with other and unspecified intracranial |
| | hemorrhage, with concussion, unspecified |
| 803.03 | Other closed skull fracture without mention of intracranial injury, with moderate |
| | [1-24 hours] loss of consciousness |
| 803.05 | Other closed skull fracture without mention of intracranial injury, with prolonged [more than 24 |
| | hours] loss of consciousness, without return to pre-existing conscious level |
| 803.1 | Other closed skull fracture with cerebral laceration and |
| | contusion |
| 803.1 | Other closed skull fracture with cerebral laceration and contusion, |
| | unspecified state of consciousness |
| 803.11 | Other closed skull fracture with cerebral laceration and contusion with no |
| | loss of consciousness |
| 803.12 | Other closed skull fracture with cerebral laceration and contusion with brief [less |
| | than one hour] loss of consciousness |
| 803.13 | Other closed skull fracture with cerebral laceration and contusion with moderate |
| | [1-24 hours] loss of consciousness |
| 803.15 | Other closed skull fracture with cerebral laceration and contusion, with prolonged [more than 24 |
| | hours] loss of consciousness, without return to pre-existing conscious level |
| 803.16 | Other closed skull fracture with cerebral laceration and contusion with loss of |
| | consciousness of unspecified duration |
| 803.19 | Other closed skull fracture with cerebral laceration and contusion, with |
| | concussion, unspecified |
| 803.2 | Other closed skull fracture with subarachnoid subdural and extradural |
| | hemorrhage |
| 803.2 | Other closed skull fracture with subarachnoid, subdural, and extradural |
| | hemorrhage, unspecified state of consciousness |
| 803.21 | Other closed skull fracture with subarachnoid, subdural, and extradural |
| | hemorrhage, with no loss of consciousness |
| 803.22 | Other closed skull fracture with subarachnoid, subdural, and extradural hemorrhage, with |
| | brief [less than one hour] loss of consciousness |
| 803.23 | Other closed skull fracture with subarachnoid, subdural, and extradural hemorrhage, |
| | with moderate [1-24 hours] loss of consciousness |
| 803.24 | Other closed skull fracture with subarachnoid, subdural, and extradural hemorrhage, with |
| | prolonged [more than 24 hours] loss of consciousness and return to pre-existing conscious level |
| 803.25 | Other closed skull fracture with subarachnoid, subdural, and extradural hemorrhage, with |
| | prolonged [more than 24 hours] loss of consciousness, without return to pre-existing conscious |
| | level |
| 803.26 | Other closed skull fracture with subarachnoid, subdural, and extradural hemorrhage, |
| | with loss of consciousness of unspecified duration |
| 803.29 | Other closed skull fracture with subarachnoid, subdural, and extradural |
| | hemorrhage, with concussion, unspecified |
| 803.3 | Closed skull fracture with other and unspecified intracranial |
| | hemorrhage |
| 803.3 | Other closed skull fracture with other and unspecified intracranial hemorrhage, |
| | unspecified state of unconsciousness |
| 803.31 | Other closed skull fracture with other and unspecified intracranial hemorrhage, |
| | with no loss of consciousness |
| 803.32 | Other closed skull fracture with other and unspecified intracranial hemorrhage, with |
| | brief [less than one hour] loss of consciousness |
| 803.33 | Other closed skull fracture with other and unspecified intracranial hemorrhage, with |
| | moderate [1-24 hours] loss of consciousness |
| 803.34 | Other closed skull fracture with other and unspecified intracranial hemorrhage, with prolonged |
| | [more than 24 hours] loss of consciousness and return to pre-existing conscious level |
| 803.35 | Other closed skull fracture with other and unspecified intracranial hemorrhage, with prolonged |
| | [more than 24 hours] loss of consciousness, without return to pre-existing conscious level |
| 803.36 | Other closed skull fracture with other and unspecified intracranial hemorrhage, with |
| | loss of consciousness of unspecified duration |
| 803.39 | Other closed skull fracture with other and unspecified intracranial |
| | hemorrhage, with concussion, unspecified |
| 803.55 | Other open skull fracture without mention of intracranial injury, with prolonged [more than 24 |
| | hours] loss of consciousness, without return to pre-existing conscious level |
| 803.6 | Other open skull fracture with cerebral laceration and contusion |
| 803.6 | Other open skull fracture with cerebral laceration and contusion, unspecified |
| | state of consciousness |
| 803.61 | Other open skull fracture with cerebral laceration and contusion, with no |
| | loss of consciousness |
| 803.63 | Other open skull fracture with cerebral laceration and contusion with moderate |
| | [1-24 hours ] loss of consciousness |
| 803.65 | Other open skull fracture with cerebral laceration and contusion, with prolonged [more than 24 |
| | hours] loss of consciousness, without return to pre-existing conscious level |
| 803.66 | Other open skull fracture with cerebral laceration and contusion, with loss of |
| | consciousness of unspecified duration |
| 803.7 | Other open skull fracture with subarachnoid subdural and extradural |
| | hemorrhage |
| 803.71 | Other open skull fracture with subarachnoid, subdural, and extradural |
| | hemorrhage, with no loss of consciousness |
| 803.72 | Other open skull fracture with subarachnoid, subdural, and extradural hemorrhage, with |
| | brief [less than one hour] loss of consciousness |
| 803.73 | Other open skull fracture with subarachnoid, subdural, and extradural hemorrhage, |
| | with moderate [1-24 hours] loss of consciousness |
| 803.74 | Other open skull fracture with subarachnoid, subdural, and extradural hemorrhage, with prolonged |
| | [more than 24 hours] loss of consciousness and return to pre-existing conscious level |
| 803.75 | Other open skull fracture with subarachnoid, subdural, and extradural hemorrhage, with prolonged |
| | [more than 24 hours] loss of consciousness, without return to pre-existing conscious level |
| 803.76 | Other open skull fracture with subarachnoid, subdural, and extradural hemorrhage, |
| | with loss of consciousness of unspecified duration |
| 803.8 | Other open skull fracture with other and unspecified intracranial |
| | hemorrhage |
| 803.81 | Other open skull fracture with other and unspecified intracranial hemorrhage |
| | with no loss of consciousness |
| 803.84 | Other open skull fracture with other and unspecified intracranial hemorrhage, with prolonged |
| | [more than 24 hours] loss of consciousness and return to pre-existing conscious level |
| 803.85 | Other open skull fracture with other and unspecified intracranial hemorrhage with prolonged [more |
| | than 24 hours] loss of consciousness, without return to pre-existing conscious level |
| 803.86 | Other open skull fracture with other and unspecified intracranial hemorrhage with |
| | loss of consciousness of unspecified duration |
| 803.89 | Other open skull fracture with other and unspecified intracranial |
| | hemorrhage, with concussion, unspecified |
| 804.03 | Closed fractures involving skull or face with other bones, without mention of intracranial |
| | injury, with moderate [1-24 hours] loss of consciousness |
| 804.04 | Closed fractures involving skull or face with other bones, without mention or intracranial injury, |
| | with prolonged [more than 24 hours] loss of consciousness and return to pre-existing conscious |
| | level |
| 804.05 | Closed fractures involving skull of face with other bones, without mention of intracranial injury, |
| | with prolonged [more than 24 hours] loss of consciousness, without return to pre-existing |
| | conscious level |
| 804.1 | Closed fractures involving skull or face with other bones with cerebral |
| | laceration and contusion |
| 804.12 | Closed fractures involving skull or face with other bones, with cerebral laceration and |
| | contusion, with brief [less than one hour] loss of consciousness |
| 804.13 | Closed fractures involving skull or face with other bones, with cerebral laceration and |
| | contusion, with moderate [1-24 hours] loss of consciousness |
| 804.14 | Closed fractures involving skull or face with other bones, with cerebral laceration and contusion, |
| | with prolonged [more than 24 hours] loss of consciousness and return to pre-existing conscious |
| | level |
| 804.16 | Closed fractures involving skull or face with other bones, with cerebral laceration and |
| | contusion, with loss of consciousness of unspecified duration |
| 804.2 | Closed fractures involving skull or face with other bones with subarachnoid |
| | subdural and extradural hemorrhage |
| 804.21 | Closed fractures involving skull or face with other bones with subarachnoid, subdural, |
| | and extradural hemorrhage, with no loss of consciousness |
| 804.22 | Closed fractures involving skull or face with other bones with subarachnoid, subdural, and |
| | extradural hemorrhage, with brief [less than one hour] loss of consciousness |
| 804.23 | Closed fractures involving skull or face with other bones with subarachnoid, subdural, and |
| | extradural hemorrhage, with moderate [1-24 hours] loss of consciousness |
| 804.24 | Closed fractures involving skull or face with other bones with subarachnoid, subdural, and |
| | extradural hemorrhage, with prolonged [more than 24 hours] loss of consciousness and return to |
| | pre-existing conscious level |
| 804.25 | Closed fractures involving skull or face with other bones with subarachnoid, subdural, and |
| | extradural hemorrhage, with prolonged [more than 24 hours] loss of consciousness, without return |
| | to pre-existing conscious level |
| 804.26 | Closed fractures involving skull or face with other bones with subarachnoid, subdural, and |
| | extradural hemorrhage, with loss of consciousness of unspecified duration |
| 804.29 | Closed fractures involving skull or face with other bones with subarachnoid, subdural, |
| | and extradural hemorrhage, with concussion, unspecified |
| 804.3 | Closed fractures involving skull or face with other bones, with other and |
| | unspecified intracranial hemorrhage |
| 804.31 | Closed fractures involving skull or face with other bones, with other and unspecified |
| | intracranial hemorrhage with no loss of consciousness |
| 804.32 | Closed fractures involving skull or face with other bones, with other and unspecified |
| | intracranial hemorrhage, with brief [less than one hour] loss of consciousness |
| 804.33 | Closed fractures involving skull or face with other bones, with other and unspecified |
| | intracranial hemorrhage, with moderate [1-24 hours] loss of consciousness |
| 804.35 | Closed fractures involving skull or face with other bones, with other and unspecified intracranial |
| | hemorrhage with prolonged [more than 24 hours] loss of consciousness, without return to pre- |
| | existing conscious level |
| 804.36 | Closed fractures involving skull or face with other bones, with other and unspecified |
| | intracranial hemorrhage, with loss of consciousness of unspecified duration |
| 804.6 | Open fractures involving skull or face with other bones, with cerebral laceration and |
| | contusion, unspecified state of consciousness |
| 804.62 | Open fractures involving skull or face with other bones, with cerebral laceration and |
| | contusion, with brief [less than one hour] loss of consciousness |
| 804.64 | Open fractures involving skull or face with other bones, with cerebral laceration and contusion, |
| | with prolonged [more than 24 hours] loss of consciousness and return to pre-existing conscious |
| | level |
| 804.7 | Open fractures involving skull or face with other bones with subarachnoid, subdural, and |
| | extradural hemorrhage, unspecified state of consciousness |
| 804.71 | Open fractures involving skull or face with other bones with subarachnoid, subdural, and |
| | extradural hemorrhage, with no loss of consciousness |
| 804.72 | Open fractures involving skull or face with other bones with subarachnoid, subdural, and |
| | extradural hemorrhage, with brief [less than one hour] loss of consciousness |
| 804.74 | Open fractures involving skull or face with other bones with subarachnoid, subdural, and |
| | extradural hemorrhage, with prolonged [more than 24 hours] loss of consciousness and return to |
| | pre-existing conscious level |
| 804.76 | Open fractures involving skull or face with other bones with subarachnoid, subdural, and |
| | extradural hemorrhage, with loss of consciousness of unspecified duration |
| 804.8 | Open fractures involving skull or face with other bones, with other and unspecified |
| | intracranial hemorrhage, unspecified state of consciousness |
| 851 | Cerebral laceration and contusion |
| 851 | Cortex (cerebral) contusion without mention of open intracranial |
| | wound |
| 851 | Cortex (cerebral) contusion without mention of open intracranial wound, |
| | unspecified state of consciousness |
| 851.01 | Cerebral laceration and contusion with no loss of consciousness |
| 851.02 | Cerebral laceration and contusion with brief [less than one hour] loss of |
| | consciousness |
| 851.03 | Cerebral laceration and contusion with moderate [1-24 hours] loss of |
| | consciousness |
| 851.04 | Cerebral laceration and contusion with prolonged [more than 24 hours] loss of |
| | consciousness and return to pre-existing conscious level |
| 851.05 | Cerebral laceration and contusion with prolonged [more than 24 hours] loss of |
| | consciousness without return to pre-existing conscious level |
| 851.06 | Cerebral laceration and contusion with loss of consciousness of |
| | unspecified duration |
| 851.09 | Cortex (cerebral) contusion without mention of open intracranial wound, |
| | with concussion, unspecified |
| 851.1 | Cortex (cerebral) contusion with open intracranial wound |
| 851.1 | Cortex (cerebral) contusion with open intracranial wound, unspecified |
| | state of consciousness |
| 851.11 | Cortex (cerebral) contusion with open intracranial wound, with no loss |
| | of consciousness |
| 851.15 | Cortex (cerebral) contusion with open intracranial wound, with prolonged [more than 24 |
| | hours] loss of consciousness without return to pre-existing conscious level |
| 851.16 | Cortex (cerebral) contusion with open intracranial wound, with loss of |
| | consciousness of unspecified duration |
| 851.2 | Cortex (cerebral) laceration without mention of open intracranial |
| | wound |
| 851.2 | Cortex (cerebral) laceration without mention of open intracranial |
| | wound |
| 851.21 | Cortex (cerebral) laceration without mention of open intracranial wound |
| | with no loss of consciousness |
| 851.22 | Cortex (cerebral) laceration without mention of open intracranial wound, with brief |
| | [less than one hour] loss of consciousness |
| 851.24 | Cortex (cerebral) laceration without mention of open intracranial wound, with prolonged [more |
| | than 24 hours] loss of consciousness and return to pre-existing conscious level |
| 851.25 | Cortex (cerebral) laceration without mention of open intracranial wound with prolonged [more |
| | than 24 hours] loss of consciousness without return to pre-existing conscious level |
| 851.26 | Cortex (cerebral) laceration without mention of open intracranial wound with loss of |
| | consciousness of unspecified duration |
| 851.29 | Cortex (cerebral) laceration without mention of open intracranial wound, |
| | with concussion, unspecified |
| 851.3 | Cortex (cerebral) laceration with open intracranial wound |
| 851.31 | Cortex (cerebral) laceration with open intracranial wound with no loss |
| | of consciousness |
| 851.34 | Cortex (cerebral) laceration with open intracranial wound, with prolonged [more than 24 |
| | hours] loss of consciousness and return to pre-existing conscious level |
| 851.35 | Cortex (cerebral) laceration with open intracranial wound with prolonged [more than 24 hours] |
| | loss of consciousness without return to pre-existing conscious level |
| 851.36 | Cortex (cerebral) laceration with open intracranial wound with loss of |
| | consciousness of unspecified duration |
| 851.4 | Cerebellar or brain stem contusion without mention of open |
| | intracranial wound |
| 851.4 | Cerebellar or brain stem contusion without mention of open intracranial wound, |
| | unspecified state of consciousness |
| 851.41 | Cerebellar or brain stem contusion without mention of open intracranial wound |
| | with no loss of consciousness |
| 851.42 | Cerebellar or brain stem contusion without mention of open intracranial wound with |
| | brief [less than one hour] loss of consciousness |
| 851.43 | Cerebellar or brain stem contusion without mention of open intracranial wound with |
| | moderate [1-24 hours] loss of consciousness |
| 851.44 | Cerebellar or brain stem contusion without mention of open intracranial wound with prolonged |
| | [more than 24 hours] loss consciousness and return to pre-existing conscious level |
| 851.45 | Cerebellar or brain stem contusion without mention of open intracranial wound with prolonged |
| | [more than 24 hours] loss of consciousness without return to pre-existing conscious level |
| 851.46 | Cerebellar or brain stem contusion without mention of open intracranial wound with |
| | loss of consciousness of unspecified duration |
| 851.49 | Cerebellar or brain stem contusion without mention of open intracranial |
| | wound with concussion, unspecified |
| 851.5 | Cerebellar or brain stem contusion with open intracranial wound, |
| | unspecified state of consciousness |
| 851.52 | Cerebellar or brain stem contusion with open intracranial wound, with brief [less |
| | than one hour] loss of consciousness |
| 851.54 | Cerebellar or brain stem contusion with open intracranial wound, with prolonged [more than 24 |
| | hours] loss of consciousness and return to pre-existing conscious level |
| 851.55 | Cerebellar or brain stem contusion with open intracranial wound, with prolonged [more than 24 |
| | hours] loss of consciousness without return to pre-existing conscious level |
| 851.56 | Cerebellar or brain stem contusion with open intracranial wound with loss of |
| | consciousness of unspecified duration |
| 851.6 | Cerebellar or brain stem laceration without mention of open |
| | intracranial wound |
| 851.61 | Cerebellar or brain stem laceration without mention of open intracranial wound, |
| | with no loss of consciousness |
| 851.62 | Cerebellar or brain stem laceration without mention of open intracranial wound, with |
| | brief [less than 1 hour] loss of consciousness |
| 851.64 | Cerebellar or brain stem laceration without mention of open intracranial wound, with prolonged |
| | [more than 24 hours] loss of consciousness and return to pre-existing conscious level |
| 851.65 | Cerebellar or brain stem laceration without mention of open intracranial wound, with prolonged |
| | [more than 24 hours] loss of consciousness without return to pre-existing conscious level |
| 851.66 | Cerebellar or brain stem laceration without mention of open intracranial wound, |
| | with concussion, unspecified |
| 851.75 | Cerebellar or brain stem laceration with open intracranial wound with prolonged [more than 24 |
| | hours] loss of consciousness without return to pre-existing conscious level |
| 851.76 | Cerebellar or brain stem laceration with open intracranial wound, with loss of |
| | consciousness of unspecified duration |
| 851.8 | Other and unspecified cerebral laceration and contusion without mention of |
| | open intracranial wound |
| 851.8 | Other and unspecified cerebral laceration and contusion, without mention of open |
| | intracranial wound, unspecified state of consciousness |
| 851.81 | Other and unspecified cerebral laceration and contusion, without mention of open |
| | intracranial wound, with no loss of consciousness |
| 851.82 | Other and unspecified cerebral laceration and contusion, without mention of open intracranial |
| | wound, with brief [less than one hour] loss of consciousness |
| 851.83 | Other and unspecified cerebral laceration and contusion, without mention of open intracranial |
| | wound, with moderate [1-24 hours] loss of consciousness |
| 851.84 | Other and unspecified cerebral laceration and contusion, without mention of open intracranial |
| | wound, with prolonged [more than 24 hours] loss of consciousness and return to pre- existing |
| | conscious level |
| 851.85 | Other and unspecified cerebral laceration and contusion, without mention of open intracranial |
| | wound, with prolonged [more than 24 hours] loss of consciousness without return to pre-existing |
| | conscious level |
| 851.86 | Other and unspecified cerebral laceration and contusion, without mention of open intracranial |
| | wound, with loss of consciousness of unspecified duration |
| 851.89 | Other and unspecified cerebral laceration and contusion, without mention of open |
| | intracranial wound, with concussion, unspecified |
| 851.9 | Other and unspecified cerebral laceration and contusion with open |
| | intracranial wound |
| 851.91 | Other and unspecified cerebral laceration and contusion, with open intracranial |
| | wound, with no loss of consciousness |
| 851.92 | Other and unspecified cerebral laceration and contusion, with open intracranial wound, |
| | with brief [less than one hour] loss of consciousness |
| 851.94 | Other and unspecified cerebral laceration and contusion, with open intracranial wound, with |
| | prolonged [more than 24 hours] loss of consciousness and return to pre-existing conscious level |
| 851.95 | Other and unspecified cerebral laceration and contusion, with open intracranial wound, with |
| | prolonged [more than 24 hours] loss of consciousness without return to pre-existing conscious |
| | level |
| 851.96 | Other and unspecified cerebral laceration and contusion, with open intracranial wound, |
| | with loss of consciousness of unspecified duration |
| 852 | Subarachnoid subdural and extradural hemorrhage following |
| | injury |
| 852 | Subarachnoid hemorrhage following injury without mention of open |
| | intracranial wound |
| 852 | Subarachnoid hemorrhage following injury without mention of open intracranial |
| | wound, unspecified state of consciousness |
| 852.01 | Subarachnoid subdural and extradural hemorrhage following injury with no |
| | loss of consciousness |
| 852.02 | Subarachnoid subdural and extradural hemorrhage following injury with brief |
| | [less than one hour] loss of consciousness |
| 852.03 | Subarachnoid subdural and extradural hemorrhage following injury with |
| | moderate [1-24 hours] loss of consciousness |
| 852.04 | Subarachnoid subdural and extradural hemorrhage following injury with prolonged [more than 24 |
| | hours] loss of consciousness and return to pre-existing conscious level |
| 852.05 | Subarachnoid subdural and extradural hemorrhage following injury with prolonged [more than 24 |
| | hours] loss of consciousness without return to pre-existing conscious level |
| 852.06 | Subarachnoid subdural and extradural hemorrhage following injury with loss of |
| | consciousness of unspecified duration |
| 852.09 | Subarachnoid subdural and extradural hemorrhage following injury with |
| | concussion, unspecified |
| 852.1 | Subarachnoid hemorrhage following injury with open intracranial |
| | wound |
| 852.1 | Subarachnoid hemorrhage following injury with open intracranial wound, |
| | unspecified state of consciousness |
| 852.11 | Subarachnoid hemorrhage following injury with open intracranial wound |
| | with no loss of consciousness |
| 852.12 | Subarachnoid hemorrhage following injury with open intracranial wound with brief |
| | [less than one hour] loss of consciousness |
| 852.13 | Subarachnoid hemorrhage following injury with open intracranial wound with |
| | moderate [1-24 hours] loss of consciousness |
| 852.15 | Subarachnoid hemorrhage following injury with open intracranial wound, with prolonged [more |
| | than 24 hours] loss of consciousness without return to pre-existing conscious level |
| 852.16 | Subarachnoid hemorrhage following injury with open intracranial wound with loss |
| | of consciousness of unspecified duration |
| 852.19 | Subarachnoid hemorrhage following injury with open intracranial wound, |
| | with concussion, unspecified |
| 852.2 | Subdural hemorrhage following injury without mention of open |
| | intracranial wound |
| 852.2 | Subdural hemorrhage following injury without mention of open intracranial |
| | wound, unspecified state of consciousness |
| 852.21 | Subdural hemorrhage following injury without mention of open intracranial |
| | wound with no loss of consciousness |
| 852.22 | Subdural hemorrhage following injury without mention of open intracranial wound |
| | with brief [less than one hour] loss of consciousness |
| 852.23 | Subdural hemorrhage following injury without mention of open intracranial wound |
| | with moderate [1-24 hours] loss of consciousness |
| 852.24 | Subdural hemorrhage following injury without mention of open intracranial wound with prolonged |
| | [more than 24 hours] loss of consciousness and return to pre-existing conscious level |
| 852.25 | Subdural hemorrhage following injury without mention of open intracranial wound with prolonged |
| | [more than 24 hours] loss of consciousness without return to pre-existing conscious level |
| 852.26 | Subdural hemorrhage following injury without mention of open intracranial wound |
| | with loss of consciousness of unspecified duration |
| 852.29 | Subdural hemorrhage following injury without mention of open intracranial |
| | wound with concussion, unspecified |
| 852.3 | Subdural hemorrhage following injury with open intracranial |
| | wound |
| 852.3 | Subdural hemorrhage following injury with open intracranial |
| | wound |
| 852.31 | Subdural hemorrhage following injury with open intracranial wound with no |
| | loss of consciousness |
| 852.32 | Subdural hemorrhage following injury with open intracranial wound, with brief |
| | [less than one hour] loss of consciousness |
| 852.33 | Subdural hemorrhage following injury with open intracranial wound, with |
| | moderate [1-24 hours] loss of consciousness |
| 852.34 | Subdural hemorrhage following injury with open intracranial wound, with prolonged [more than |
| | 24 hours] loss of consciousness and return to pre-existing conscious level |
| 852.35 | Subdural hemorrhage following injury with open intracranial wound, with prolonged [more than |
| | 24 hours] loss of consciousness without return to pre-existing conscious level |
| 852.36 | Subdural hemorrhage following injury with open intracranial wound with loss of |
| | consciousness of unspecified duration |
| 852.4 | Extradural hemorrhage following injury without mention of open |
| | intracranial wound |
| 852.4 | Extradural hemorrhage following injury without mention of open intracranial |
| | wound, unspecified state of consciousness |
| 852.41 | Extradural hemorrhage following injury without mention of open intracranial |
| | wound with no loss of consciousness |
| 852.42 | Extradural hemorrhage following injury without mention of open intracranial wound |
| | with brief [less than 1 hour] loss of consciousness |
| 852.43 | Extradural hemorrhage following injury without mention of open intracranial wound |
| | with moderate [1-24 hours] loss of consciousness |
| 852.44 | Extradural hemorrhage following injury without mention of open intracranial wound with |
| | prolonged [more than 24 hours] loss of consciousness and return to pre-existing conscious level |
| 852.45 | Extradural hemorrhage following injury without mention of open intracranial wound with |
| | prolonged [more than 24 hours] loss of consciousness without return to pre-existing conscious |
| | level |
| 852.46 | Extradural hemorrhage following injury without mention of open intracranial wound |
| | with loss of consciousness of unspecified duration |
| 852.49 | Extradural hemorrhage following injury without mention of open intracranial |
| | wound with concussion, unspecified |
| 852.5 | Extradural hemorrhage following injury with open intracranial |
| | wound |
| 852.5 | Extradural hemorrhage following injury with open intracranial wound, |
| | unspecified state of consciousness |
| 852.51 | Extradural hemorrhage following injury with open intracranial wound with |
| | no loss of consciousness |
| 852.54 | Extradural hemorrhage following injury with open intracranial wound, with prolonged [more than |
| | 24 hours] loss of consciousness and return to pre-existing conscious level |
| 852.56 | Extradural hemorrhage following injury with open intracranial wound, with loss |
| | of consciousness of unspecified duration |
| 853 | Other and unspecified intracranial hemorrhage following injury |
| 853 | Other and unspecified intracranial hemorrhage following injury without |
| | mention of open intracranial wound |
| 853 | Other and unspecified intracranial hemorrhage following injury without mention of open |
| | intracranial wound, unspecified state of consciousness |
| 853.01 | Other and unspecified intracranial hemorrhage following injury without mention of open |
| | intracranial wound, with no loss of consciousness |
| 853.02 | Other and unspecified intracranial hemorrhage following injury without mention of open |
| | intracranial wound, with brief [less than one hour] loss of consciousness |
| 853.03 | Other and unspecified intracranial hemorrhage following injury without mention of open |
| | intracranial wound, with moderate [1-24 hours] loss of consciousness |
| 853.04 | Other and unspecified intracranial hemorrhage following injury without mention of open |
| | intracranial wound, with prolonged [more than 24 hours] loss of consciousness and return to pre- |
| | existing conscious level |
| 853.05 | Other and unspecified intracranial hemorrhage following injury without mention of open |
| | intracranial wound, with prolonged [more than 24 hours] loss of consciousness without return to |
| | pre-existing conscious level |
| 853.06 | Other and unspecified intracranial hemorrhage following injury without mention of open |
| | intracranial wound, with loss of consciousness of unspecified duration |
| 853.09 | Other and unspecified intracranial hemorrhage following injury without mention of open |
| | intracranial wound, with concussion, unspecified |
| 853.1 | Other and unspecified intracranial hemorrhage following injury with |
| | open intracranial wound |
| 853.1 | Other and unspecified intracranial hemorrhage following injury with open |
| | intracranial wound, unspecified state of consciousness |
| 853.11 | Other and unspecified intracranial hemorrhage following injury with open |
| | intracranial wound with no loss of consciousness |
| 853.13 | Other and unspecified intracranial hemorrhage following injury with open intracranial |
| | wound, with moderate [1-24 hours] loss of consciousness |
| 853.15 | Other and unspecified intracranial hemorrhage following injury with open intracranial wound, with |
| | prolonged [more than 24 hours] loss of consciousness without return to pre-existing conscious |
| | level |
| 853.16 | Other and unspecified intracranial hemorrhage following injury with open intracranial |
| | wound with loss of consciousness of unspecified duration |

### Genotyping

Genotyping was performed as previously described.[2] CHOP patients were genotyped on a mixture of Illumina SNP arrays (HumanHap 550, 610 Quad, OmniExpress, and GSA arrays).

### Genome Wide Association

Prior to genotype imputation, the data was filtered for sample call rate>95%, SNP call rate>95%, minor allele frequency>1% and Hardy-Weinberg equilibrium p>1×10-6 using Plink 1.9 on the world wide web at .cog-genomics.org/plink/1.9/[3]. The genome-wide Complex Trait Analysis (GCTA) program was used to calculate eigenvectors for all samples.[4] Then a k-nearest neighbor's algorithm was used to assign samples to their respective genetic ancestries as previously described. [5] Only patients with European or African ancestry were included in the GWAS.

All CHOP genotyping data was imputed to the HRC v1.1 reference panel using either the Sanger imputation server or the Michigan imputation server.[6] The genotyping data was separated by chip type and ancestral group. The data was pre-phased prior to imputation using EAGLE2. [7] Three principal components were used as covariates from a PCA analysis to control for population stratification. SAIGE was used for association in each cohort to control for case-control imbalances and a meta-regression (MR-MEGA) meta-analysis was used for a trans-ancestral analysis. [1][8] One principal component was used in the meta-regression in MR-MEGA. GWAMA was used for meta-analysis within ancestral groups. [9] Genomic control (GC) correction was used for each cohort before meta-analysis and for the full meta-analysis results.

Cohchrans Q-statistic and i2 were examined in the GWAMA meta-analysis results to estimate heterogeneity between studies.

Manhattan plots and quantile-quantile plots were made within R using the package CMplot (https://github.com/YinLiLin/CMplot).

### Proxy SNPs

LDlink LDproxy tool was used to identify proxies of SNPs. [10] The 1000 genome project panel was used with build hg38 to have the most recent panel. [11] The panel was limited to the population with African Ancestry in Southwest US to have the LD most like the CHOP African ancestry population.

### Expression QTLs

GTEx Analysis Release V8 (dbGaP Accession phs000424.v8.p2) single tissue cis-egtl data was downloaded and significant variant-gene associations based on permutations were searched to find an overlap between significant eqtl associations and the list of proxy SNPs.

### Example 1

### Identification of SNPS Associated with Increased Risk for mTBI

### Discovery Meta-Analysis

The CHOP cohorts were initially meta-analyzed as a trans-ancestral discovery cohort. Double GC correction was used in MR-MEGA, and no individual cohort had a GC value above 1.068. The GC of the full meta-analysis was 1.014. There was only one locus, on chromosome 4, that was genome wide significant (Index SNP rs1470721, p=3.41×10⁻⁸) in this discovery cohort. Table 2 shows the genome wide significant results of the MR-MEGA analysis for the trans-ancestral CHOP meta-analysis.

TABLE 2

| | | | | | FIXED [symbol] ONLY | | | | | | | | |
|---|---|---|---|---|---|---|---|---|---|---|---|---|---|
| | | | Effect | Other | Effect Allele | | | | | [symbol] | | [symbol] | |
| [symbol] | Chromosome | [symbol] | Allele | Allele | Frequency | Beta | [symbol] | [symbol] | [symbol] | [symbol] | [symbol] | [symbol] | [symbol] |
| rs [symbol] | 4 | [symbol] | A | [symbol] | 0.75 | −0.30 | [symbol] | [symbol] | 0 | [symbol] | [symbol] | [symbol] | [symbol] |
| rs [symbol] | 4 | [symbol] | [symbol] | [symbol] | 0.74 | −0.30 | [symbol] | [symbol] | 0 | [symbol] | [symbol] | [symbol] | [symbol] |
| rs [symbol] | 4 | [symbol] | C | [symbol] | 0.75 | −0.30 | [symbol] | [symbol] | 0 | [symbol] | [symbol] | [symbol] | [symbol] |
| rs [symbol] | 4 | [symbol] | A | [symbol] | 0.75 | [symbol] | [symbol] | [symbol] | 0 | [symbol] | [symbol] | [symbol] | [symbol] |
| rs [symbol] | 4 | [symbol] | C | [symbol] | 0.75 | [symbol] | [symbol] | [symbol] | 0 | [symbol] | [symbol] | [symbol] | [symbol] |
| rs [symbol] | 4 | [symbol] | [symbol] | [symbol] | 0.75 | −0.32 | [symbol] | [symbol] | 0 | [symbol] | [symbol] | [symbol] | [symbol] |
| rs [symbol] | 4 | [symbol] | A | [symbol] | 0.75 | −0.32 | [symbol] | [symbol] | 0 | [symbol] | [symbol] | [symbol] | [symbol] |
| rs [symbol] | 4 | [symbol] | A | [symbol] | 0.75 | −0.30 | [symbol] | [symbol] | 0 | [symbol] | [symbol] | [symbol] | [symbol] |
| rs [symbol] | 4 | [symbol] | [symbol] | [symbol] | 0.75 | [symbol] | [symbol] | [symbol] | 0 | [symbol] | [symbol] | [symbol] | [symbol] |
| rs [symbol] | 4 | [symbol] | [symbol] | A | 0.75 | [symbol] | [symbol] | [symbol] | 0 | [symbol] | [symbol] | [symbol] | [symbol] |

[symbol]

indicates data missing or illegible when filed

The SNPs listed in Table 2 are present in SEQ ID NOS: 1-10 in descending order in the table. The effect allele is listed in the table and is at 75% AF with negative beta value, thus the effects are protective and the “other” alleles are in column 5 are the risk alleles.

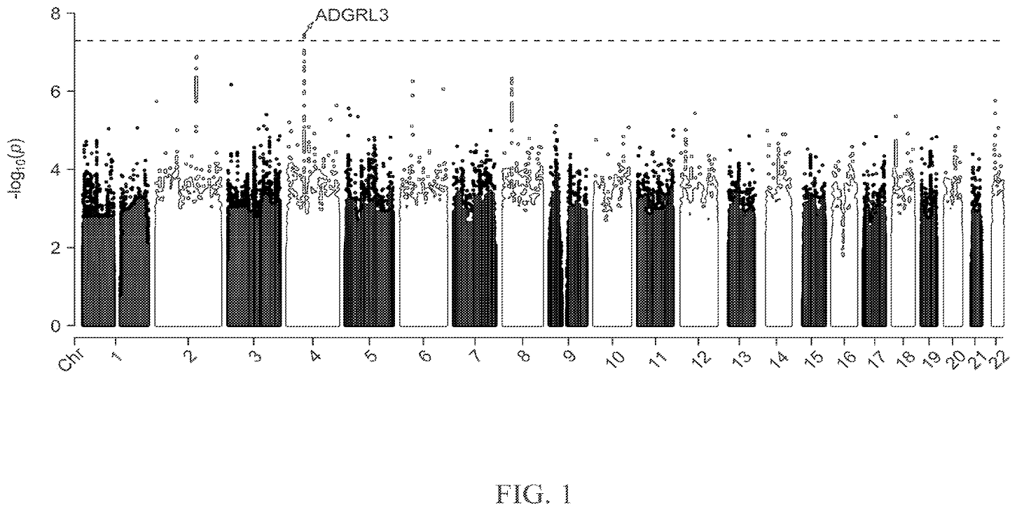

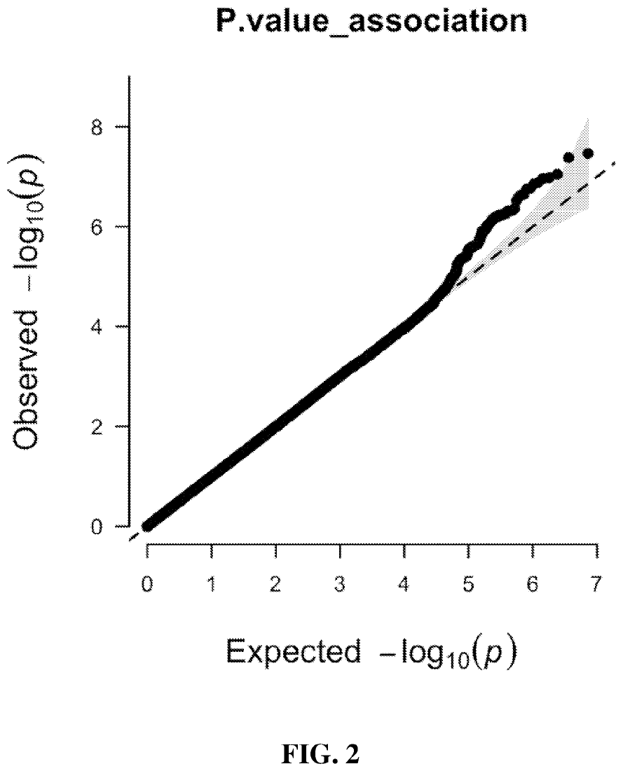

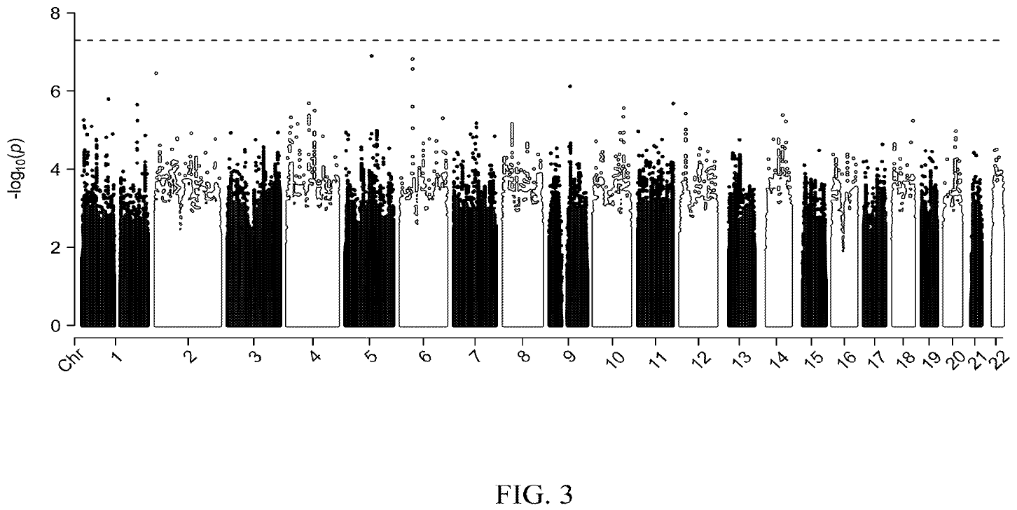

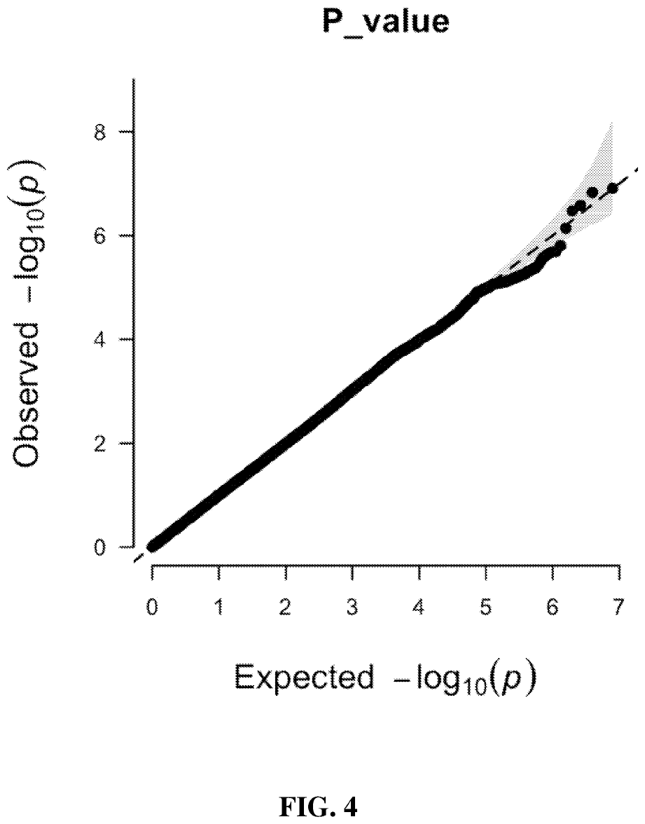

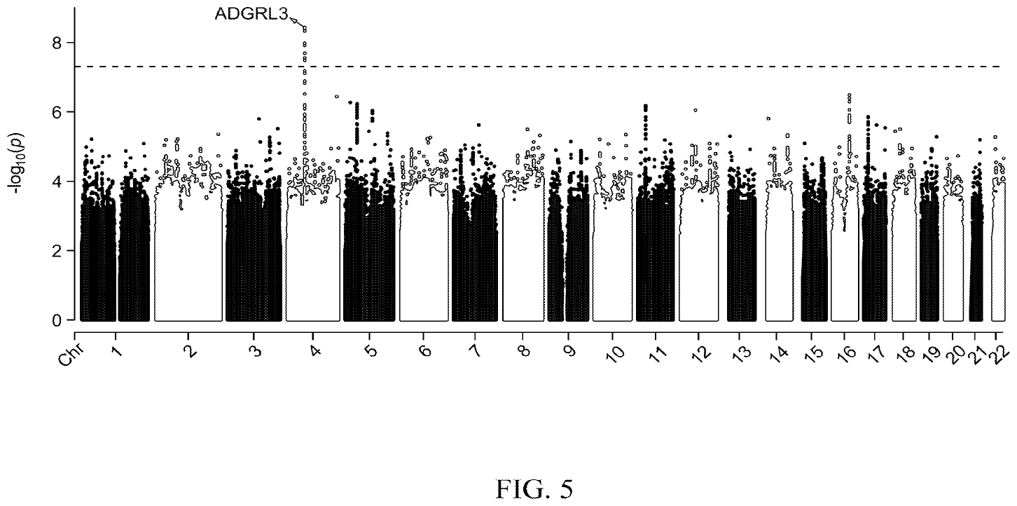

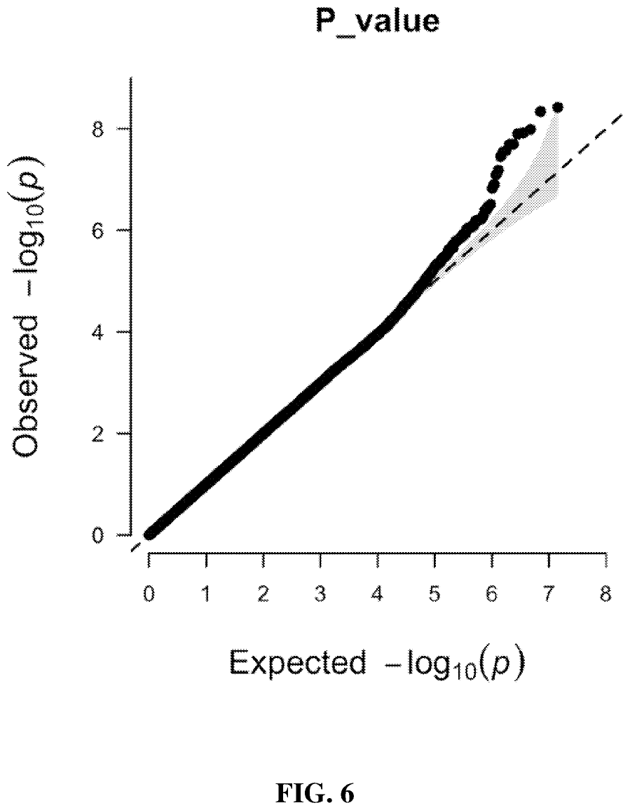

FIG. 1 and FIG. 2 show the Manhattan plot and quantile-quantile plot for the CHOP trans-ancestral meta-analysis respectively. There was a significant ancestral heterogeneity component to the meta-analysis (p=3.124x⁻⁶), so we analyzed each ancestry separately using Genome-Wide Association Meta-Analysis (GWAMA) to see the contributions from each ancestry. FIG. 3 and FIG. 4 shows the Manhattan plot and quantile-quantile plot for the CHOP European meta-analysis respectively. The index SNP was not significant (p=0.95) in the CHOP meta-analysis using only European American samples, but it was even more significant (p=3.79×10⁻⁹) in the CHOP meta-analysis using only the African American samples. Table 2 shows the genome wide significant results from the African American fixed effects meta-analysis utilizing only CHOP patients. FIG. 5 and FIG. 6 shows the Manhattan plot and quantile-quantile plot for the CHOP African meta-analysis respectively.

### Replication in Biobanks

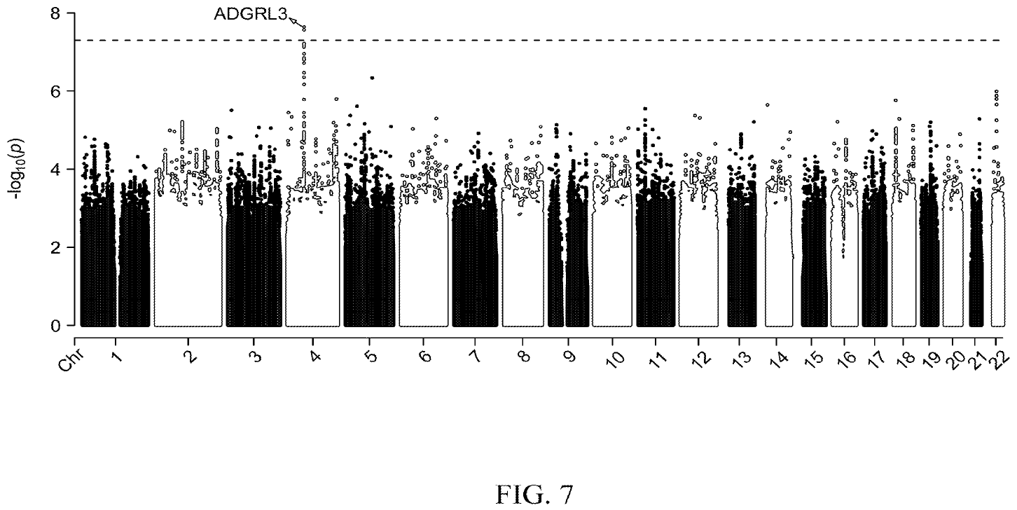

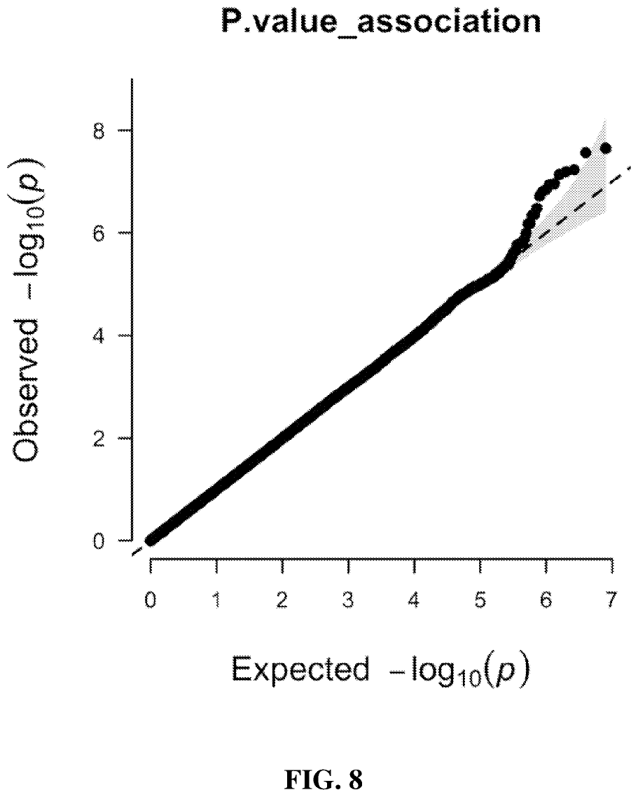

Even though this association was being driven by the African American samples, we proceeded with another meta-analysis involving the UK BioBank and the Finngen Biobank. While the index SNP didn't reach the significance (p=2.26×10⁻⁸) of the African American meta-analysis, it did get more significant than the CHOP only trans-ancestral meta-analysis. FIG. 7 and FIG. 8 show the Manhattan plot and quantile-quantile plot for the full meta-analysis respectively.

### Locus Functional Identity

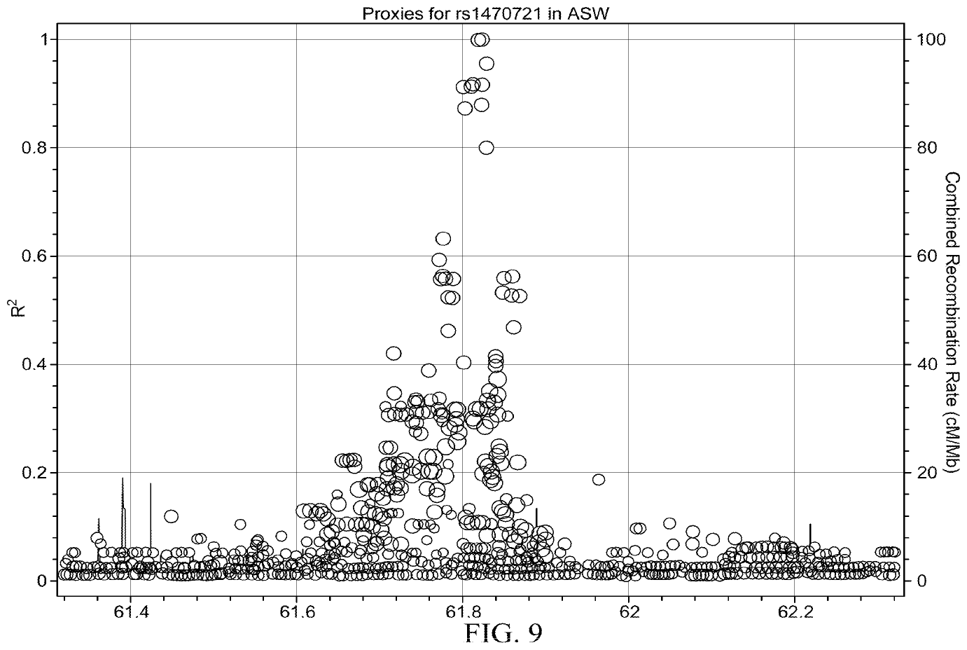

To try to identify potential causal SNPs, all proxies (r2>0.2) of the index SNP (rs1470721) were found in the 1000 genomes project ASW population using LDlink. FIG. 9 shows the proxies and the recombination rate in the population in the region. The index SNP (rs1470721) at the locus is intronic to latrophilin 3 (ADGRL3). All the proxies lie between position 61,655,668 bases to 61,867,719 bases on chromosome 4 on build hg38. All the variants are either intronic or exonic to the gene ADGRL3. Seven of the 179 proxy SNPs showed a significant (p<2.86×10⁻⁴) association with ADGRL3 in thyroid tissue. One of these SNPs (rs10434219) is also a synonymous coding variant of ADGRL3. The index SNP also shows nominal significance (p=0.046) as an eqtl to ADGRL3 in brain anterior cingulate cortex (Brodmann area 24) but this is not considered significant when multiple testing is taken into account.

### Example 2

### Screening Assays for Identifying Efficacious Therapeutics for the Treatment of mTBI or Concussion

The information herein above can be applied clinically to patients for diagnosing an increased susceptibility for developing mTBI and therapeutic intervention. An embodiment of the invention comprises clinical application of the information described herein to a patient. Diagnostic compositions, including microarrays, and methods can be designed to identify the SNPs described herein in nucleic acids from a patient to assess susceptibility for developing mTBI. This can occur after a patient arrives in the clinic; the patient has blood drawn, and using the diagnostic methods described herein, a clinician can detect a genetic alteration such as a single nucleotide polymorphism as described in Example 1. The information obtained from the patient sample, which can optionally be amplified prior to assessment, may be used to diagnose a patient with an increased or decreased susceptibility for developing mTBI or used to direct treatment in a patient previously diagnosed with mTBI. Kits for performing diagnostic methods of the invention are also provided herein. Such kits may comprise a microarray comprising at least one of the SNPs provided herein and the necessary reagents for assessing the patient samples as described above.

The identity of mTBI involved genes and the patient results will indicate which variants are present, and may be used to identify those that have, or possess an altered risk for developing mTBI. The information provided herein may allow for therapeutic intervention at earlier times in disease progression than previously possible. Also as described herein above, the genes listed in Table 2 herein were shown to associate with mTBI at genome wide significance (GWS) levels. Thus, the latrophilin-3 gene provides a novel target for the development of new therapeutic agents efficacious for the treatment of mTBI.

### Example 3

### Test and Treat Method for Ameliorating Symptoms Associated with mTBI

In order to treat an individual having mTBI for example, to alleviate a sign or symptom of the mTBI, suitable agents targeting the ADGRL3 (also known as latrophilin-3) gene disclosed herein can be administered in combination in order to provide therapeutic benefit to the patient. Such agents should be administered in an effective dose.

First, a biological sample, or genotyping information would be obtained from a patient. Genetic information gleaned from nucleic acids present in the sample would then be assessed for the presence or absence of the mTBI-associated SNP containing nucleic acids associated with increased risk of mTBI. The presence of these SNPs indicating the presence of risk for mTBI, providing the clinician with guidance as to which therapeutic agents or prophylactic methods are appropriate. The total treatment dose or doses (when two or more targets are to be modulated) can be administered to a subject as a single dose or can be administered using a fractionated treatment protocol, in which multiple/separate doses are administered over a more prolonged period of time, for example, over the period of a day to allow administration of a daily dosage or over a longer period of time to administer a dose over a desired period of time. One skilled in the art would know that the amount of mTBI agent required to obtain an effective dose in a subject depends on many factors, including the age, weight and general health of the subject, as well as the route of administration and the number of treatments to be administered. In view of these factors, the skilled artisan would adjust the particular dose so as to obtain an effective dose for treating an individual having mTBI.

The effective dose of mTBI therapeutic agent(s) will depend on the mode of administration, and the weight of the individual being treated. The dosages described herein are generally those for an average adult but can be adjusted for the treatment of children. The dose will generally range from about 0.001 mg to about 1000 mg.

Administration of the pharmaceutical preparation is preferably in an “effective amount” this being sufficient to show benefit to the individual. This amount prevents, alleviates, abates, or otherwise reduces the severity of at least one mTBI symptom in a patient.

Methods for the safe and effective administration of FDA-approved pharmaceutical gluatamatergic and ionotropic modulating agents are known to those skilled in the art. Table 1 provides such agents that should have efficacy for the treatment of mTBI. The racetam family of agents, particularly fasoracetam, are preferred for this purpose. In addition, their administration is described in the standard literature. For example, the administration of many anti-inflammatory agents is described in the “Physicians' Desk Reference” (PDR), e.g., 1996 edition (Medical Economics Company, Montvale, N.J. 07645-1742, USA); the disclosure of which is incorporated herein by reference thereto.

TABLE 3
N-methyl D-aspartate receptors (NMDARS)
Negative Allosteric Modulators (NAMs)

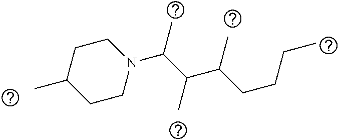 [SMILES: CCCCC(C)C(C)C(C)N1CCC(C)CC1]

CP-101806,
Teanaprodil

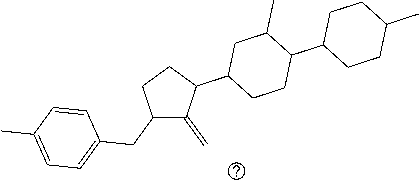

BMS-986169

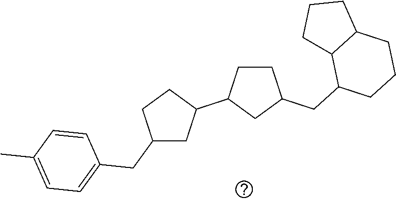 [SMILES: Cc1ccc(CC2CCC(C3CCC(CC4CCCC5CCCC54)C3)C2)cc1]

MK-22

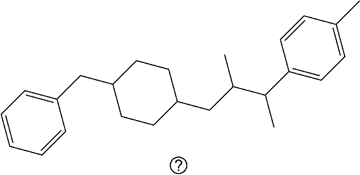 [SMILES: Cc1ccc(C(C)C(C)CC2CCC(Cc3ccccc3)CC2)cc1]

Ru3S-6981

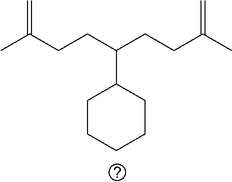 [SMILES: C=C(C)CCC(CCC(=C)C)C1CCCCC1]

Felbanoate

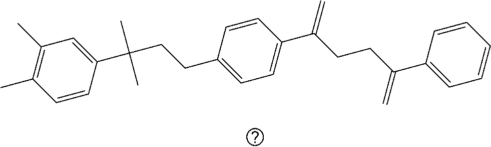 [SMILES: C=C(CCC(=C)c1ccc(CCC(C)(C)c2ccc(C)c(C)c2)cc1)c1ccccc1]

TCN-201

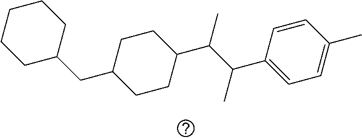 [SMILES: Cc1ccc(C(C)C(C)C2CCC(CC3CCCCC3)CC2)cc1]

Ifenprodil

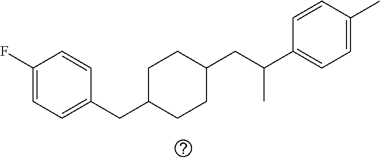 [SMILES: Cc1ccc(C(C)CC2CCC(Cc3ccc(F)cc3)CC2)cc1]

SL-820735, Eliprodil

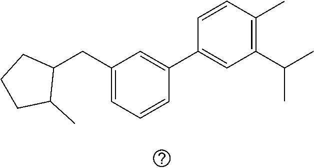 [SMILES: Cc1ccc(-c2cccc(CC3CCCC3C)c2)cc1C(C)C]

EVT-101

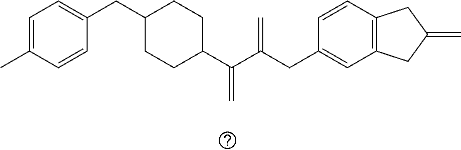

Radiprodil

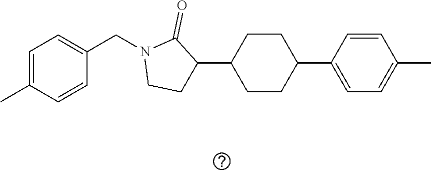

BMT-108998

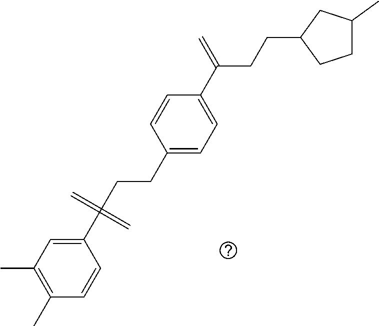

MPX-004

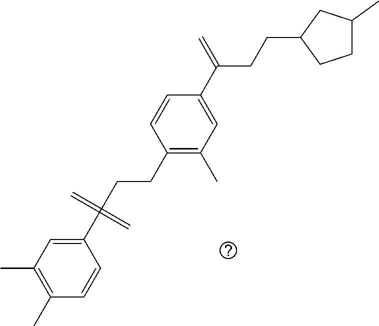

MPX-007

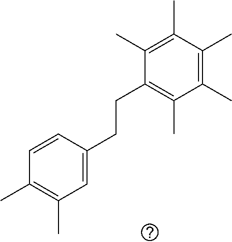

Xeo200

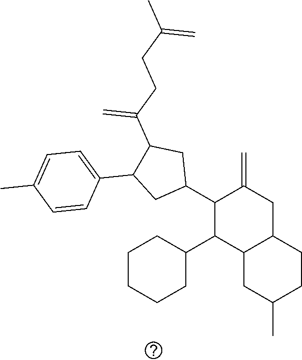 [SMILES: C=C(C)CCC(=C)C1CC(C2C(=C)CC3CCC(C)CC3C2C2CCCCC2)CC1c1ccc(C)cc1]

DQP-1105

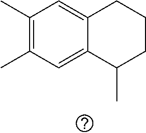

GBPS51

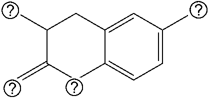

UBP608

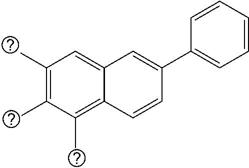 [SMILES: Cc1cc2cc(-c3ccccc3)ccc2c(C)c1C]

UBP618

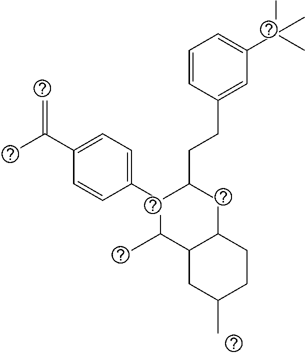 [SMILES: C=C(C)c1ccc(C2C(CCc3cccc(C(C)(C)C)c3)CC3CCC(C)CC3C2C)cc1]

QNZ46
Positive Allosteric Modulators (PAMs)

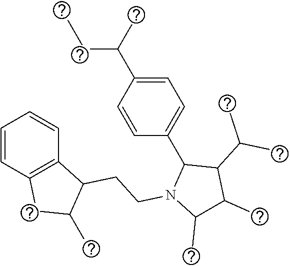 [SMILES: CCC(C)c1ccc(C2C(C(C)C)C(C)C(C)N2CCC2c3ccccc3CC2C)cc1]

PYD-106

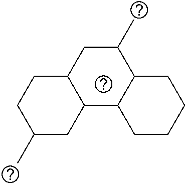 [SMILES: CC1CCC2CC(C)C3CCCCC3C2C1]

UBP512

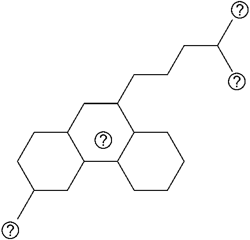 [SMILES: CC(C)CCCC1CC2CCC(C)CC2C2CCCCC12]

UBP646

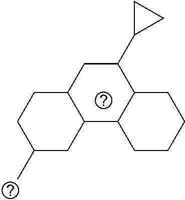 [SMILES: CC1CCC2CC(C3CC3)C3CCCCC3C2C1]

UBP710

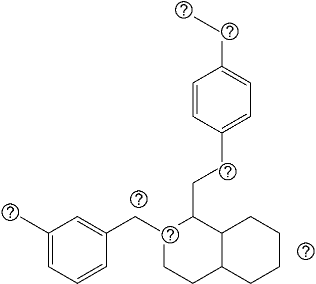 [SMILES: CCc1ccc(CCC2C(Cc3cccc(C)c3)CCC3CCCCC32)cc1]

CIQ

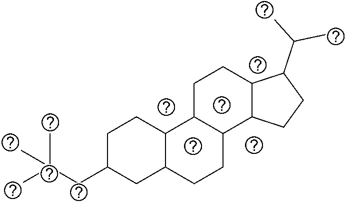 [SMILES: CC(C)C1CCC2C1CCC1C3CCC(CC(C)(C)C)CC3CCC12]

Pregnenzolone sulfate

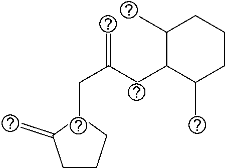 [SMILES: C=C(CC1CCCC1=C)CC1C(C)CCCC1C]

Nelfracetam

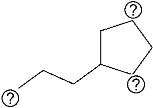

Histamine

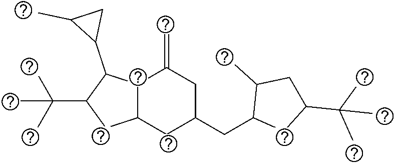 [SMILES: C=C1CC(CC2CC(C(C)(C)C)CC2C)CC2CC(C(C)(C)C)C(C3CC3C)C12]

GNE-0723

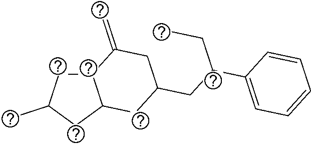 [SMILES: C=C1CC(CC(CC)c2ccccc2)CC2CC(C)CC12]

GNE-3419

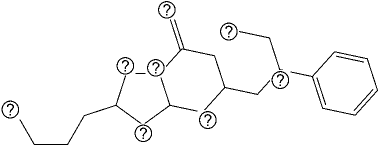 [SMILES: C=C1CC(CC(CC)c2ccccc2)CC2CC(CCCC)CC12]

GNE-3476

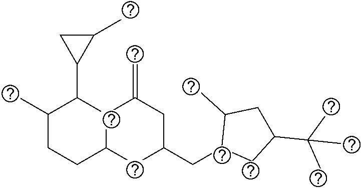 [SMILES: C=C1CC(CC2CC(C(C)(C)C)CC2C)CC2CCC(C)C(C3CC3C)C12]

GNE-5729

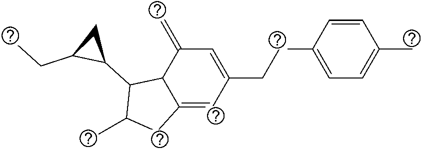 [SMILES: C=C1C=C(CCc2ccc(C)cc2)C=C2CC(C)C([C@@H]3C[C@@H]3CC)C12]

GNE-6901

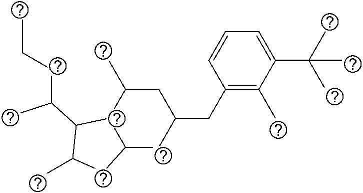 [SMILES: CCCC(C)C1C(C)CC2CC(Cc3cccc(C(C)(C)C)c3C)CC(C)C21]

GNE-8018

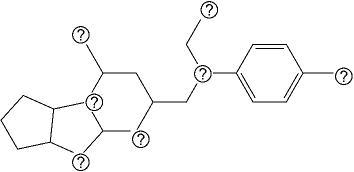 [SMILES: CCC(CC1CC(C)C2C(C1)CC1CCCC12)c1ccc(C)cc1]

GNE-8324

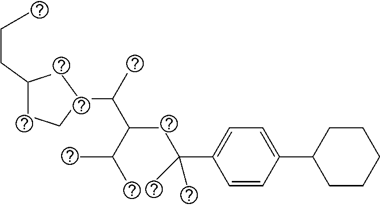 [SMILES: CCCC1CCC(C(C)C(CC(C)(C)c2ccc(C3CCCCC3)cc2)C(C)C)C1]

GNE-9278

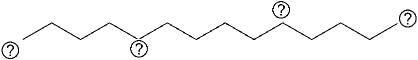

Spenantine

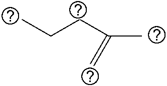

D-serine
(S)-2-Amino-3-(5-methyl-3-hydroxybenzaxol-4-yl)-propanoic acid receptors (AMPARs)
Negative Allosteric Modulators (NAMs)

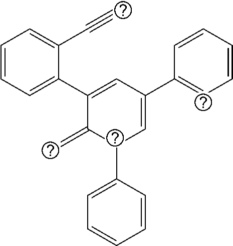 [SMILES: C#Cc1ccccc1C1=CC(c2ccccc2)=CC(c2ccccc2)C1=C]

Perampanel, PMP

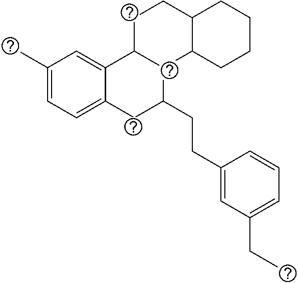 [SMILES: CCc1cccc(CCC2Cc3ccc(C)cc3C3CCC4CCCCC4C23)c1]

CP-465022

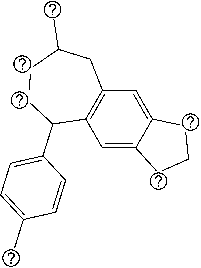 [SMILES: Cc1ccc(C2CCC(C)Cc3cc4c(cc32)CCC4)cc1]

GYKI-52466

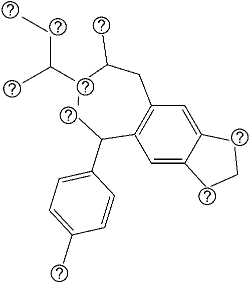 [SMILES: CCC(C)C1CC(c2ccc(C)cc2)c2cc3c(cc2CC1C)CCC3]

GYKI-53655

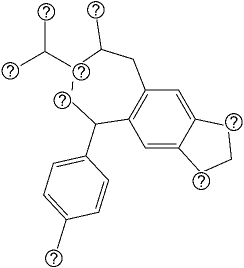 [SMILES: Cc1ccc(C2CC(C(C)C)C(C)Cc3cc4c(cc32)CCC4)cc1]

Talampanel, GYKI-53773

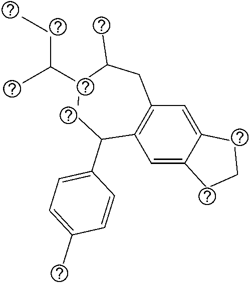 [SMILES: CCC(C)C1CC(c2ccc(C)cc2)c2cc3c(cc2CC1C)CCC3]

GYKI-53784
Positive Allosteric Modulators (PAMs)

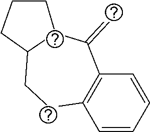

ORG-28576

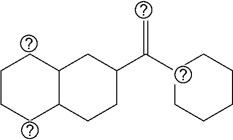 [SMILES: C=C(C1CCCCC1)C1CCC2CCCCC2C1]

CX 516, Ampalex

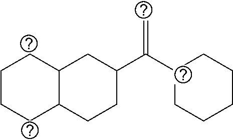 [SMILES: C=C(C1CCCCC1)C1CCC2CCCCC2C1]

CX516

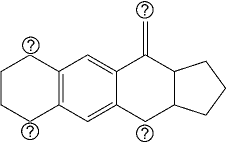 [SMILES: C=C1c2cc3c(cc2CC2CCCC12)CCCC3]

CX614

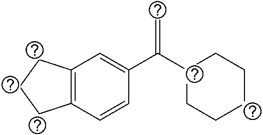 [SMILES: C=C(c1ccc2c(c1)CCC2)C1CCCCC1]

CX 691,
Farampator

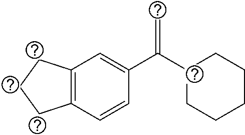 [SMILES: C=C(c1ccc2c(c1)CCC2)C1CCCCC1]

CX717

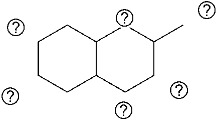

Cyclothiazole (CYZ)

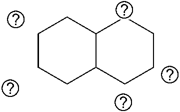

Hydrochlorothiazole

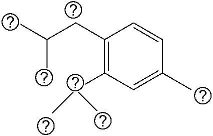

IDRA-21

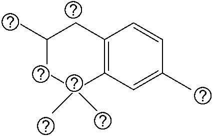

Diaxozide
Proglycan

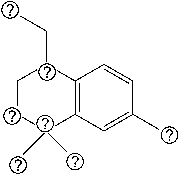

BPAM97

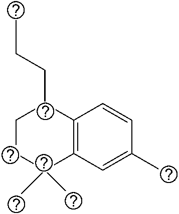 [SMILES: CCCC1CCC(C)(C)c2cc(C)ccc21]

BPAM125

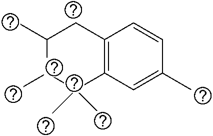

BPAM321

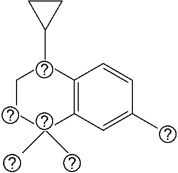 [SMILES: Cc1ccc2c(c1)C(C)(C)CCC2C1CC1]

BPAM344

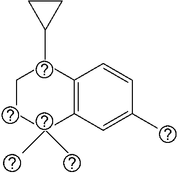 [SMILES: Cc1ccc2c(c1)C(C)(C)CCC2C1CC1]

BPAM523

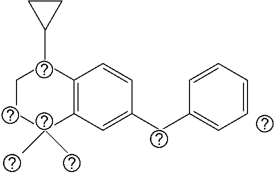 [SMILES: CC1(C)CCC(C2CC2)c2ccc(Cc3ccccc3)cc21]

BPAM538

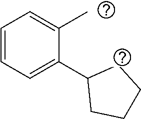

SS5986

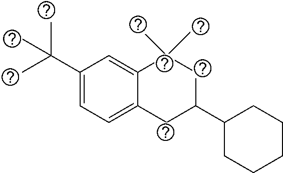 [SMILES: CC(C)(C)c1ccc2c(c1)C(C)(C)CC(C1CCCCC1)C2]

SS3376

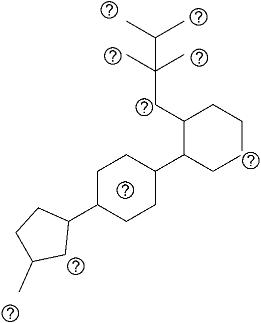 [SMILES: CC1CCC(C2CCC(C3CCCCC3CC(C)(C)C(C)C)CC2)C1]

SS-74

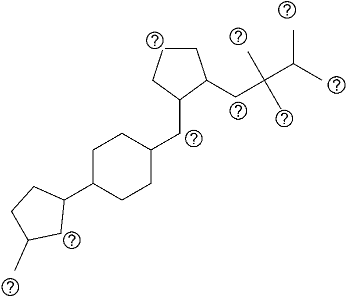 [SMILES: CC1CCC(C2CCC(CC3CCCC3CC(C)(C)C(C)C)CC2)C1]

SS-04958242

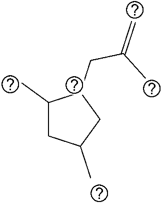

Ostracetam

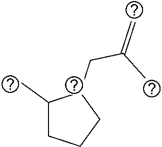

Piracetam

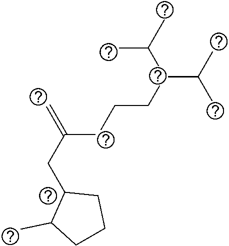 [SMILES: C=C(CCCC(C(C)C)C(C)C)CC1CCCC1C]

Pramiracetam

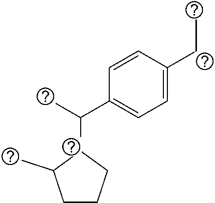 [SMILES: CCc1ccc(C(C)C2CCCC2C)cc1]

Antracetam

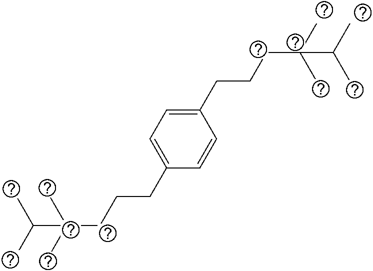 [SMILES: CC(C)C(C)(C)CCCc1ccc(CCCC(C)(C)C(C)C)cc1]

CMPDA

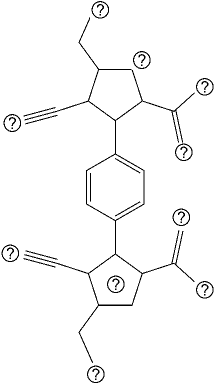 [SMILES: C#CC1C(CC)CC(C(=C)C)C1c1ccc(C2C(C(=C)C)CC(CC)C2C#C)cc1]

CMPDO

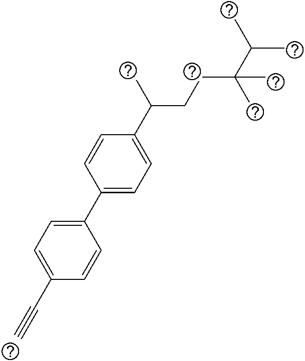 [SMILES: C#Cc1ccc(-c2ccc(C(C)CCC(C)(C)C(C)C)cc2)cc1]

LY404187

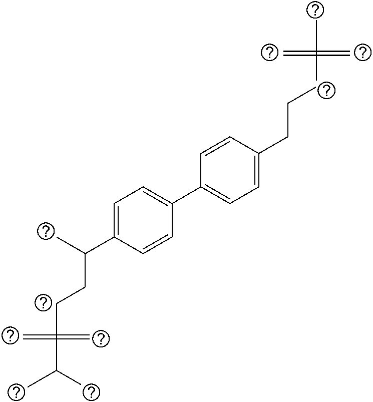

Alihampator, LY458395

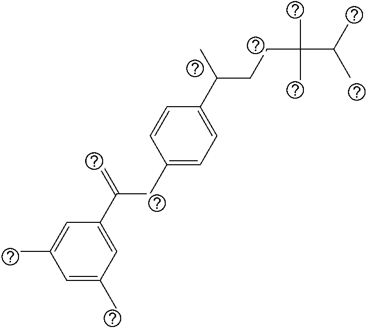 [SMILES: C=C(Cc1ccc(C(C)CCC(C)(C)C(C)C)cc1)c1cc(C)cc(C)c1]

LY450106

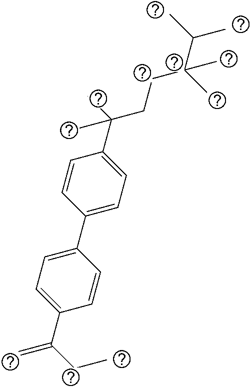 [SMILES: C=C(CC)c1ccc(-c2ccc(C(C)(C)CCC(C)(C)C(C)C)cc2)cc1]

LY503430

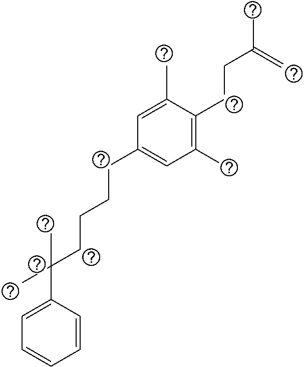 [SMILES: C=C(C)CCc1c(C)cc(CCCCC(C)(C)c2ccccc2)cc1C]

PEPA

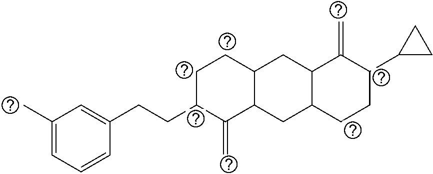 [SMILES: C=C1C(CCc2cccc(C)c2)CCC2CC3C(=C)C(C4CC4)CCC3CC12]

54744S, Talamprofen

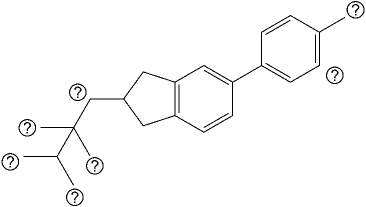 [SMILES: Cc1ccc(-c2ccc3c(c2)CC(CC(C)(C)C(C)C)C3)cc1]

GSK729327

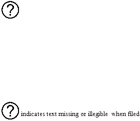

The present invention also encompasses a pharmaceutical composition useful in the treatment of mTBI, comprising the administration of a therapeutically effective amount of the combinations of this invention, with or without pharmaceutically acceptable carriers or diluents. The compositions of the present invention may further comprise one or more pharmaceutically acceptable additional ingredient(s) such as alum, stabilizers, antimicrobial agents, buffers, coloring agents, flavoring agents, adjuvants, and the like. The anti-mTBI compositions of the present invention may be administered orally or parenterally including the intravenous, intramuscular, intraperitoneal, subcutaneous, rectal and topical routes of administration.

Determination of the proper dosage for a particular situation is routine in the clinical arts. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

In certain methods, the subject is tested prior to participating in sports associated with frequent mTBI injuries. Detection of the mTBI-associated SNPs could be followed by treatment with glutamatergic and ionotropic agents prophylactically throughout the sport season.

As described above in Example I, genome wide association studies (GWAS) have identified ADGRL3 as a susceptibility gene associated with mTBI in patients of African Ancestry. ADGRL3 is a G protein-coupled receptor associated with attention deficit hyperactivity disorder and human cognitive function that we for the first time are reporting as a genome-wide significant locus in children and adolescents with concussion. Taken together, these findings suggest that the glutamatergic modulator, fasoracetam and related racetam compounds, may be an effective therapy for patients who have sustained concussion injury.

### REFERENCES

[1] Zhou W, Nielsen J B, Fritsche L G, Dey R, Gabrielsen M E, Wolford B N, LeFaive J, VandeHaar P, Gagliano S A, Gifford A, Bastarache L A, Wei W Q, Denny J C, Lin M, Hveem K, Kang H M, Abecasis G R, Willer C J, Lee S. Efficiently controlling for case-control imbalance and sample relatedness in large-scale genetic association studies. Nat Genet. 2018 September; 50(9):1335-1341. doi: 10.1038/s41588-018-0184-y. Epub 2018 August 13. PMID: 30104761; PMCID: PMC6119127.
[2] Hakonarson H, Grant S F, Bradfield J P, Marchand L, Kim C E, Glessner J T, Grabs R, Casalunovo T, Taback S P, Frackelton E C, Lawson M L, Robinson L J, Skraban R, Lu Y, Chiavacci R M, Stanley C A, Kirsch S E, Rappaport E F, Orange J S, Monos D S, Devoto M, Qu H Q, Polychronakos C. A genome-wide association study identifies KIAA0350 as a type 1 diabetes gene. Nature. 2007 Aug. 2; 448(7153):591-4. doi: 10.1038/nature06010. Epub 2007 July 15. PMID: 17632545.
[3] Chang C C, Chow C C, Tellier L C, Vattikuti S, Purcell S M, Lee J J. Second-generation PLINK: rising to the challenge of larger and richer datasets. Gigascience. 2015 Feb. 25; 4:7. doi: 10.1186/s13742-015-0047-8. PMID: 25722852; PMCID: PMC4342193.
[4] Yang J, Lee S H, Goddard M E, Visscher P M. GCTA: a tool for genome-wide complex trait analysis. Am J Hum Genet. 2011 Jan. 7; 88(1):76-82. doi: 10.1016/j.ajhg.2010.11.011. Epub 2010 December 17. PMID: 21167468; PMCID: PMC3014363.
[5] Buono R J, Bradfield J P, Wei Z, Sperling M R, Dlugos D J, Privitera M D, French J A, Lo W, Cossette P, Schachter S C, Basehore H, Lohoff F W, Grant S F A, Ferraro T N, Hakonarson H. Genetic Variation in PADI6-PADI4 on 1p36.13 Is Associated with Common Forms of Human Generalized Epilepsy. Genes (Basel). 2021 Sep. 18; 12(9):1441. doi: 10.3390/genes12091441. PMID: 34573423; PMCID: PMC8472138.
[6] McCarthy S, Das S, Kretzschmar W, Delaneau O, Wood A R, Teumer A, Kang H M, Fuchsberger C, Danecek P, Sharp K, Luo Y, Sidore C, Kwong A, Timpson N, Koskinen S, Vrieze S, Scott L J, Zhang H, Mahajan A, Veldink J, Peters U, Pato C, van Duijn C M, Gillies C E, Gandin I, Mezzavilla M, Gilly A, Cocca M, Traglia M, Angius A, Barrett J C, Boomsma D, Branham K, Breen G, Brummett C M, Busonero F, Campbell H, Chan A, Chen S, Chew E, Collins F S, Corbin L J, Smith G D, Dedoussis G, Dorr M, Farmaki A E, Ferrucci L, Forer L, Fraser R M, Gabriel S, Levy S, Groop L, Harrison T, Hattersley A, Holmen O L, Hveem K, Kretzler M, Lee J C, McGue M, Meitinger T, Melzer D, Min J L, Mohlke K L, Vincent J B, Nauck M, Nickerson D, Palotie A, Pato M, Pirastu N, McInnis M, Richards J B, Sala C, Salomaa V, Schlessinger D, Schoenherr S, Slagboom P E, Small K, Spector T, Stambolian D. Tuke M, Tuomilehto J, Van den Berg L H, Van Rheenen W, Volker U, Wijmenga C, Toniolo D, Zeggini E, Gasparini P, Sampson M G, Wilson J F, Frayling T, de Bakker P I, Swertz M A, McCarroll S, Kooperberg C, Dekker A, Altshuler D, Willer C, Iacono W, Ripatti S, Soranzo N, Walter K, Swaroop A, Cucca F, Anderson C A, Myers R M, Boehnke M, McCarthy M I, Durbin R; Haplotype Reference Consortium. A reference panel of 64,976 haplotypes for genotype imputation. Nat Genet. 2016 October; 48(10):1279-83. doi: 10.1038/ng.3643. Epub 2016 August 22. PMID: 27548312; PMCID: PMC5388176.
[7] Loh P R, Danecek P, Palamara P F, Fuchsberger C, A Reshef Y, K Finucane H, Schoenherr S, Forer L, McCarthy S, Abecasis G R, Durbin R, L Price A. Reference-based phasing using the Haplotype Reference Consortium panel. Nat Genet. 2016 November; 48(11):1443-1448. doi: 10.1038/ng.3679. Epub 2016 October 3. PMID: 27694958; PMCID: PMC5096458.
[8] Magi R, Horikoshi M, Sofer T, Mahajan A, Kitajima H, Franceschini N, McCarthy M I; COGENT-Kidney Consortium, T2D-GENES Consortium, Morris A P. Trans-ethnic meta-regression of genome-wide association studies accounting for ancestry increases power for discovery and improves fine-mapping resolution. Hum Mol Genet. 2017 Sep. 15; 26(18):3639-3650. doi: 10.1093/hmg/ddx280. PMID: 28911207; PMCID: PMC5755684.
[9] Magi R, Morris A P. GWAMA: software for genome-wide association meta-analysis. BMC Bioinformatics. 2010 May 28; 11:288. doi: 10.1186/1471-2105-11-288. PMID: 20509871; PMCID: PMC2893603.
[10] Machiela M J, Chanock S J. LDlink: a web-based application for exploring population-specific haplotype structure and linking correlated alleles of possible functional variants. Bioinformatics. 2015 Nov. 1; 31(21):3555-7. doi: 10.1093/bioinformatics/btv402. Epub 2015 July 2. PMID: 26139635; PMCID: PMC4626747.
[11]1000 Genomes Project Consortium, Auton A, Brooks L D, Durbin R M, Garrison E P, Kang H M, Korbel J O, Marchini J L, McCarthy S, McVean G A, Abecasis G R. A global reference for human genetic variation. Nature. 2015 Oct. 1; 526(7571):68-74. doi: 10.1038/nature15393. PMID: 26432245; PMCID: PMC4750478.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

What is claimed is:

1. A method for identifying a subject at increased risk for concussion or mild traumatic brain injury (mTBI), comprising
a) detecting in a biological sample obtained from a subject suspected of having or being at increased risk for concussion or mTBI, a nucleic acid encoding the risk allele in present in the ADGRL3 gene listed in Table 2; and
b) diagnosing said subject as being at an increased risk for mTBI or concussion.

2. The method of claim 1, wherein said nucleic acid encodes the risk allele present in rs1470721 and listed in Table 2.

3. The method of claim 1, wherein said plasma sample is derived from said subject within about 0.1 to about 8 hours of a head injury.

4. The method of claim 1, wherein the step of detecting in the nucleic acid a single nucleotide polymorphism (SNP) in said ADGRL3 gene comprises performance of a process selected from the group consisting of detection of specific hybridization, measurement of allele size, restriction fragment length polymorphism analysis, allele-specific hybridization analysis, single base primer extension reaction, and sequencing of an amplified polynucleotide.

5. The method of claim 4, wherein said process is performed on a solid support.

6. The method of claim 5, wherein said solid support comprises a strip, a glass, a silicon, a polymer, a bead, or a nanoparticle.

7. A method of treating concussion or mild traumatic brain injury (mTBI) with an agent that modulates ADGRL3 activity, comprising:
a) detecting in a biological sample obtained from a subject suspected of having or being at increased risk for concussion or mTBI, a nucleic acid encoding one or more SNPs comprising risk alleles present in the ADGRL3 gene listed in Table 2;
b) diagnosing said subject as being at an increased risk for mTBI or concussion.
c) administering to said subject a therapeutically effective amount of a compound that modulates the activity or level of ADGRL3, thereby treating said subject.

8. The method of claim 7, wherein said agent is listed in Table 4.

9. The method of claim 7, wherein said compound comprises a glutamatergic modulator or ionotropic modulator listed in Table 3.

10. The method of claim 7, wherein said nucleic acid encodes the risk allele present in rs1470721.

11. The method of claim 7, wherein said glutamatergic modulator is fasoracetam or a pharmaceutically acceptable salt, ester, prodrug, metabolite, polymorph or solvate thereof.

12. A method of treating concussion or mTBI in a subject, comprising administrating to said subject a therapeutically effective amount of fasoracetam to said subject, thereby alleviating at least one symptom of mTBI.

13. A method for protecting a subject from mTBI, comprising
a) detecting in a biological sample obtained from a subject suspected of having or being at increased risk for concussion or mTBI, a nucleic acid encoding one or more SNPs comprising risk alleles present in the ADGRL3 gene listed in Table 2; and
b) administering an effective amount of a glutamatergic or ionotropic modulator to said subject, prior to participation in an activity associated with a high incidence of mTBI, thereby reducing the risk of mTBI in said subject.
